(19) United States
(12) Patent Application Publication
(10) Pub. No.: US 2014/0030244 A1
(43) Pub. Date: Jan. 30, 2014

(54) EXTRACTS ISOLATED FROM ELECTROPORATED AMBHIBIAN OOCYTES AND USE THEREOF IN TREATING DISEASES AND DISORDERS

(71) Applicant: BIOQUARK, INC., Tampa, FL (US)

(72) Inventor: Sergei Paylian, Tampa, FL (US)

(73) Assignee: BIOQUARK, INC., Tampa, FL (US)

(21) Appl. No.: 13/949,977

(22) Filed: Jul. 24, 2013

Related U.S. Application Data
(60) Provisional application No. 61/741,822, filed on Jul. 27, 2012.

(51) Int. Cl. A61K 35/56; A61K 31/7105; A61K 38/52; A61K 38/45; A61K 38/46

(52) U.S. Cl. CPC A61K 35/65; A61K 38/45; A61K 38/46; A61K 38/52; A61K 31/7105
USPC 424/94.5; 424/561; 424/94.6

(57) ABSTRACT

The described invention provides methods for preparing a composition containing extracts of activated amphibian oocytes, the method where the composition is a pharmaceutical composition comprising an equal volume of the extra-oocyte composition and the intra-oocyte composition, and a method for treating a disease, disorder, condition or injury characterized by a damaged or a cancerous differentiated cell including: (a) preparing the composition by the described method; (b) formulating a pharmaceutical composition comprising an equal volume of the extra-oocyte composition and the intra-oocyte composition, and optionally a carrier; and (c) administering a therapeutic amount of the pharmaceutical composition of (b) to a subject in need thereof, where the therapeutic amount is effective to reprogram the damaged or cancerous cells into iPSC-like cells capable of differentiating into cells capable of repairing the damaged or cancerous cells, thereby treating the disease, disorder, injury or condition.

### CROSS-REFERENCE TO RELATED APPLICATION

This application claims the benefit of priority from U.S. provisional patent application Ser. No. 61/741,822, filed Jul. 27, 2012, the entire disclosure of which is incorporated herein by reference.

### FIELD OF INVENTION

The described invention relates to cellular reprogramming; pharmaceutical compositions for cellular reprogramming of differentiated cells containing extracts isolated from electroporated amphibian oocytes, and use of such pharmaceutical compositions in regenerative medicine.

### BACKGROUND OF THE INVENTION

Human disease results from loss of organ function. Whether tissue failure results from infarction, infection, trauma, or congenital malfunction, the ideal treatment would be regrowth of a new organ or tissue to replace that which is lost or injured (See, Alonso L. and Fuchs E., Genes Dev., 2003; 17:1189-1200). Cell therapy is the transplantation of live cells into an organism in order to repair tissue or restore lost or defective functions (See, Liras A., Journal of Translational Medicine, 2010; 8:131-145). Stem cells are used for cell therapy because of their capability for unlimited self-renewal when cultured and their ability to differentiate into the specific cells required for repairing damaged or defective tissues or cells (See, Medvedev S. P. et al., Acta Naturae, 2010; 2(2):18-27 and Ahrlund-Richter L. et al., Cell Stem Cell, 2009; 4:20-26). Four classes of stem cells have been considered for use in cell therapy: (1) embryonic stem cells (ESCs); (2) adult stem cells (ASCs); (3) umbilical cord stem cells (UCSCs); and (4) induced pluripotent stem cells (iPSCs).

### Embryonic Stem Cells (ESCs)

ESCs are isolated from the inner cell mass of pre-implantation embryos (See, Thomson J. A. et al., Science, 1998; 282:1145-1147). ESCs are pluripotent (i.e., capable of differentiating into virtually every cell type), easy to isolate, and highly reproductive in culture (See, Liras A., Journal of Translational Medicine, 2010; 8:131-145). However, ESCs are an allogeneic cell source and thus are prone to immunorejection. Immunosuppressive drug regimens have been employed to lessen the severity of the immune reaction, but these regimens simultaneously place the recipient at an increased risk of infection. The use of ESCs further provide the disadvantages of possibly differentiating into inadequate cell types or of inducing tumors, as well as the ethical concerns relating to the use of human embryos for ESC derivation (See, e.g., Jung Y. et al., Stem Cells, 2012; 30:42-47 and Liras A., Journal of Translational Medicine, 2010; 8:131-145).

### Adult Stem Cells (ASCs)

Adult stem cells (ASCs) are undifferentiated cells occurring in tissues and organs of adult individuals, which can give rise to cells of the tissues and organs from which they originate (i.e., they are multipotent). For example, ASCs of the central nervous system differentiate into neurons, oligodendrocytes and astrocytes (See, Liras A., Journal of Translational Medicine, 2010; 8:131-145). ASCs occur in most tissues, including bone marrow, adipose tissue, breast gland, gastrointestinal tract, central nervous system, lung, peripheral blood, dermis and the like. ASCs hold several advantages over ESCs. For example, the use of ASCs involves autologous transplantation (i.e., the donor and recipient are the same individual), a method less likely to induce immune rejection reactions. The use of ASCs also poses no ethical concerns, since these cells are derived from adult tissues and organs. However, ASCs are difficult to isolate, grow slowly, differentiate poorly in culture, are difficult to produce in adequate amounts for transplantation, behave differently depending on the tissue source, show telomere shorting, and often carry the genetic abnormalities inherited or acquired by the donor (See, e.g., Liras A., Journal of Translational Medicine, 2010; 8:131-145).

### Umbilical Cord Stem Cells (UCSCs)

Umbilical cord stem cells (UCSCs) are a source of hematopoietic stem cells and progenitor cells for the treatment of a variety of malignant and non-malignant disorders, including acute and chronic myeloid and lymphoid leukemias, myelodysplastic syndromes, solid tumors, bone marrow failures, hemoglobinopathies, metabolic disorders, leukodystrophies and primary immunodeficiencies (See, Broxmeyer H. E., Cord Blood Hematopoietic Stem Cell Transplantation, StemBook, Copyright 2013 by the Massachusetts General Hospital, Copyright 2008-2009 by the President and Fellows of Harvard University, ISSN1940-3429). UCSCs hold an advantage over both ESCs and ASCs in that UCSCs are readily available through cord blood banks. However, the disadvantages of using UCSCs include, but are not limited to, a limiting numbers of cells collected from a single donor which can be suboptimal for transplantation, the slow speed of engraftment of neutrophils and platelets, and immune rejection reactions associated with the use of multiple cord blood units (See, Broxmeyer H. E., Cord Blood Hematopoietic Stem Cell Transplantation, StemBook, Copyright 2013 by the Massachusetts General Hospital, Copyright 2008-2009 by the President and Fellows of Harvard University, ISSN1940-3429).

Induced Pluripotent Stem Cells (iPSCs)

In 2006, it was reported that adult somatic cells could be reprogrammed from fully differentiated cells back to pluripotent stem cells by retroviral delivery of four transcription factors (Oct4, Sox2, Klf4 and Myc) (See, Takahashi K. and Yamanaka S., Cell, 2006; 126:663-76). These cells, referred to as induced pluripotent stem cells or iPSCs, closely resemble ESCs in a broad spectrum of features. For example, iPSCs have the ability to differentiate or mature into the three primary groups of cells that form a human being: (i) ectoderm cells (cells that form the skin and nervous system); (ii) endoderm cells (cells that form the gastrointestinal tract, the respiratory tract, the liver, the pancreas and the endocrine glands); and (iii) mesoderm cells (cells that form bone, cartilage, muscle, connective tissue and the circulatory system). (See, Cox J. L. and Rizzino A., Experimental Biology and Medicine, 2010; 235:148-158). iPSCs and ESCs also share similar morphologies and growth characteristics and are equally sensitive to growth factors and signaling molecules. Like ESCs, iPSCS are easy to isolate and highly reproductive in culture, an advantage both ESCs and iPSCs hold over ASCs. However, unlike both ESCs and UCSCs, iPSCs are autologous and thus are not prone to immune-rejection. The use of iPSCs can further provide the advantage of a normal, stable karyotype within established iPS cells, an advantage iPSCs hold over both ESCs and ASCs. The use of iPSCs also bypasses the ethical issues surrounding the derivation and use of ESCs to cure disease (See, e.g., Jung Y. et al., Stem Cells, 2012; 30:42-47 and Amabile A. and Meissner A, Trends in Molecular Medicine, 2009; 15(2):59-68). Therefore, iPSCs are theoretically an ideal autologous cell source for use in cell therapies designed to treat chronic debilitating diseases that have escaped remedial measures from traditional allopathic approaches.

A number of different approaches have been devised to reprogram somatic cells into iPSCs. These approaches involve the shuttling of reprogramming factors into somatic cells. Such reprogramming factor delivery methods include: (i) integrating methods; (ii) excisable methods; (iii) nonintegrating methods; and (iv) DNA-free methods.

### Integrating Methods

The first studies on iPSCs used constitutively active retroviral vectors that stably integrated into the host cell genome to introduce four genes, c-Myc, Klf4, Oct4 and Sox2, the minimal core set of genes required to generate iPSCs (See, Takahashi and Yamanaka, Cell, 2006; 126:663-76 and Stadtfeld M. and Hochedlinger K., Genes Dev., 2010; 24:2239-2263). However, incomplete silencing of retroviral transgenes often results in partially reprogrammed cells that depend on exogenous factor expression and fail to activate the corresponding endogenous genes (See, Takahashi and Yamanaka, Cell, 2006; 126:663-76; and Stadtfeld M. and Hochedlinger K., Genes Dev., 2010; 24:2239-2263). In addition, residual activity or reactivation of viral transgenes interferes with the developmental potential of iPSCs and frequently leads to tumor formation (See, Stadtfeld M. and Hochedlinger K., Genes Dev., 2010; 24:2239-2263; and Okita K. et al., Nature, 2007; 448:313-317). The risk of continued transgene expression is exacerbated when less-efficiently silenced constitutively active lentiviral vectors are used (See, Stadtfeld M. and Hochedlinger K., Genes Dev., 2010; 24:2239-2263; and Brambrink T. et al., Cell Stem Cell, 2008; 2:151-159). Continued transgene expression has been diminished by the use of inducible lentiviral vectors (See, Stadtfeld M. and Hochedlinger K., Genes Dev., 2010; 24:2239-2263; and Brambrink T. et al. Cell Stem Cell, 2008; 2:151-159). However, inducible lentiviral systems have the disadvantage of requiring multiple integrations and transactivator expression (See, Stadtfeld M. and Hochedlinger K. Genes Dev., 2010; 24:2239-2263).

### Excisable Methods

Cre protein is a site-specific DNA recombinase that can catalyze recombination of DNA between specific sites in the DNA of cells. These specific sites are known as LoxP sequences. Several laboratories have developed gene delivery vectors with incorporated loxP sites that can be excised from the host genome by transient expression of Cre recombinase (See, Stadtfeld M. and Hochedlinger K. Genes Dev., 2010; 24:2239-2263; and Kaji K. et al., Nature, 2009; 458:771-775). Vectors with incorporated loxP sites enable the efficient generation of iPSCs from diverse cell types, especially when polycistronic vectors are employed (See, Stadtfeld M. and Hochedlinger K. Genes Dev., 2010; 24:2239-2263; and Chang C. W. et al., Stem Cells, 2009; 27:1042-1049). However, short vector sequences which remain in the host cell DNA after excision can affect cellular function (See, Stadtfeld M. and Hochedlinger K. Genes Dev., 2010; 24:2239-2263).

Inducible pluripotent stem cells also have been generated with transposons. These mobile genetic elements can be introduced into and removed from the host genome by the transient expression of transposase (See, Stadtfeld M. and Hochedlinger K. Genes Dev., 2010; 24:2239-2263; and Woltjen K. et al., Nature, 2009; 458:766-770). Although the low error rate of this approach provides for a seamless excision, laborious characterization of integration sites in iPSCs before and after transposon removal is required. The expression of transposase also can induce nonspecific alterations in the iPSC genome (See, Stadtfeld M. and Hochedlinger K. Genes Dev., 2010; 24:2239-2263).

### Nonintegrating Methods

Integration-free iPSCs have been generated using adenoviral vectors, plasmids, polycistronic mini-circle vectors and self-replicating selectable episomes (See, Stadtfeld M. and Hochedlinger K. Genes Dev., 2010; 24:2239-2263; Stadtfeld M. et al., Science, 2008; 322:945-949; Okita K. et al., Science, 2008; 322:949-953; Jia F. et al., Nat Methods, 2010; 7:197-199; and Yu J. et al., Science, 2009; 324:797-801). These approaches have several disadvantages, including a low efficiency of iPSC generation (˜0.001%) and occasional vector integration into the host genome (See, Stadtfeld M. and Hochedlinger K. Genes Dev., 2010; 24:2239-2263).

### DNA-Free Methods

Reprogramming of somatic cells also has been achieved without the use of viral vectors or plasmids. For example, iPSCs have been derived by delivering reprogramming factors as purified recombinant proteins or as whole-cell extracts isolated from either embryonic stem cells or human embryonic kidney 293 (HEK293) cells (See, Stadtfeld M. and Hochedlinger K. Genes Dev., 2010; 24:2239-2263; Zhou H. et al., Cell Stem Cell, 2009; 4:381-384; Cho H. J. et al., Blood, 2010; 116:386-395; and Kim D. et al., Cell Stem Cell, 2009; 4:472-476). However, the efficiency of iPSCs generation by these approaches is low (0.001%) and in the case of the recombinant protein approach, the addition of a histone deacetylase inhibitor is required (See, Stadtfeld M. and Hochedlinger K. Genes Dev., 2010; 24:2239-2263).

Likewise, iPSCs have been created by chemical compounds that promote reprogramming. A number of compounds have been identified that promote the overexpression of c-Myc, Klf4, Oct4 and Sox2 in somatic cells (See, Stadtfeld M. and Hochedlinger K. Genes Dev., 2010; 24:2239-2263; Desponts C. and Ding S., Methods Mol Biol, 2010; 636:207-218; and Li W. and Ding S., Trends Pharmacol Sci, 2010; 31:36-45). Although providing a reasonable efficiency in the generation of iPSCs (˜0.1-1%), these chemical compounds, many of which are known modulators of DNA and chromatin modification, act to decrease the number of iPSC clones generated while introducing genetic or epigenetic abnormalities into resultant iPSCs. See, Stadtfeld M. and Hochedlinger K. Genes Dev., 2010; 24:2239-2263.

Thus, the need exists to develop an efficient method to produce cells that have the properties of iPSCs but that are free from genetic or epigenetic abnormalities and useful for therapeutic applications. The described invention provides a method for the non-viral reprogramming of damaged and cancerous differentiated cells by administering a composition comprising a therapeutic amount of an extract of activated amphibian oocytes comprising microRNAs and proteins, which is effective to reprogram the damaged and cancerous cells into iPSC-like cells.

### SUMMARY OF THE INVENTION

The present disclosure provides methods for preparing a composition containing extracts of activated amphibian oocytes and methods for treating a disease, disorder, condition or injury characterized by a damaged or a cancerous differentiated cell.

According to one aspect, the described invention provides a method for preparing a composition comprising extracts of activated amphibian oocytes comprising: (a) providing a suspension of oocytes harvested from an amphibian, in a buffered oocyte washing solution in an oocyte activation vessel; (b) applying an electroporation stimulus to the suspended oocytes of (a) in the oocyte activation vessel to produce a suspension of activated oocytes; (c) combining an aqueous energy solution with the suspension of activated oocytes to form an aqueous suspension; (d) incubating the aqueous suspension of (c) at an incubation temperature of 16° C. to 20° C., for an incubation time of about 2 to about 4 hours; (e) partitioning the incubated combination of (d) to obtain a portion external to the incubated activated oocytes (extra-oocyte portion), and an activated oocyte portion that includes the incubated activated oocytes of (d); (f) separating the extra-oocyte portion and the activated oocyte portion from each other; (g) filtering the extra-oocyte portion to produce an extra-oocyte composition; (h) rupturing the activated oocyte portion of (f) comprising a light fraction, a heavy fraction and a cytoplasmic fraction; (i) separating the cytoplasmic fraction from the light fraction and the heavy fraction to produce a combination of the light fraction and the heavy fraction; and (j) filtering the combination of (i) to obtain an intra-oocyte composition.

According to another aspect, the described invention provides a method for treating a disease, disorder, condition or injury characterized by a damaged or cancerous differentiated cell comprising: (a) preparing a composition by: (1) providing a suspension of oocytes harvested from an amphibian, in a buffered oocyte washing solution in an oocyte activation vessel; (2) applying an electroporation stimulus to the suspended oocytes of (1) in the oocyte activation vessel to produce a suspension of activated oocytes; (3) combining an aqueous energy solution with the suspension of activated oocytes to form an aqueous suspension; (4) incubating the aqueous suspension of (3) at an incubation temperature of 16° C. to 20° C., for an incubation time of about 2 to about 4 hours; (5) partitioning the incubated combination of (4) to obtain a portion external to the incubated activated oocytes (extra-oocyte portion), and an activated oocyte portion that includes the incubated activated oocytes of (4); (6) separating the extra-oocyte portion and the activated oocyte portion from each other; (7) filtering the extra-oocyte portion to produce an extra-oocyte composition; (8) rupturing the activated oocyte portion of (6) to produce a light fraction, a heavy fraction and a cytoplasmic fraction; (9) separating the cytoplasmic fraction from the light fraction and the heavy fraction to produce a combination of the light fraction and the heavy fraction; and (10) filtering the combination of (9) to obtain an intra-oocyte composition; (b) formulating a pharmaceutical composition comprising an equal volume of the extra-oocyte composition and the intra-oocyte composition, and optionally a carrier; and (c) administering a therapeutic amount of the pharmaceutical composition of (b) to a subject in need thereof, wherein the therapeutic amount is effective to reprogram the damaged or cancerous cells into iPSC-like cells capable of differentiating into cells capable of repairing the damaged or cancerous cells, thereby treating the disease, disorder, injury or condition.

According to one embodiment, the amphibian oocytes are Xenopus laevis oocytes.

According to one embodiment, the activation vessel is selected from the group consisting of a cell culture flask and an electroporation cuvette.

According to one embodiment, the electroporation stimulus is about 100 v/cm to about 200 v/cm at about 25 μF to about 75 μF for about 0.3 msec to about 1.5 msec pulses for about 5 to 10 pulses. According to another embodiment, the electroporation stimulus is about 125 v/cm at about 50 μF for about 1 msec pulses at about 7 pulses.

According to one embodiment, the incubation temperature is 17° C.

According to one embodiment, the incubation time is 3 hours.

According to one embodiment, the light fraction comprises lipids.

According to one embodiment, the heavy fraction comprises yolk particles.

According to one embodiment, the buffered oocyte washing solution comprises NaCl, HEPES, KCl, MgCl2, NaHPO4 and penicillin/streptomycin. According to another embodiment, the buffered oocyte washing solution is about pH 7.4. According to anther embodiment, the buffered oocyte washing solution comprises about 82.5 mM NaCl, about 5 mM HEPES, about 2.5 mM KCl, about 1 mM MgCl₂, about 1 mM NaHPO4 and about 0.5% penicillin/streptomycin.

According to one embodiment, the aqueous energy solution comprises creatine phosphate, adenosine-5′-triphosphate (ATP), and MgCl₂. According to another embodiment, the aqueous energy solution comprises about 7.5 mM creatine phosphate, about 1 mM adenosine-5′-triphosphate (ATP) at pH 7.7, and about 1 mM MgCl₂. According to another embodiment, the aqueous energy solution is a 1:100 aqueous dilution.

According to one embodiment, the partitioning step is performed by centrifugation.

According to one embodiment, the separating step is performed by a syringe.

According to one embodiment, the filtering step is performed by a filter. According to another embodiment, the filter has a pore size of about 0.01μ to 1μ. According to another embodiment, the filter has a pore size of about 0.2μ.

According to one embodiment, the rupturing step is performed by centrifugation.

According to one embodiment, the method further comprises combining the extra-oocyte portion with a mixture comprising a protease inhibitor and a RNase inhibitor.

According to one embodiment, the method further comprises the step of combining the light fraction and the heavy fraction combination with a protease inhibitor and a RNase inhibitor.

According to one embodiment, the composition is a pharmaceutical composition comprising an equal volume of the extra-oocyte composition and the intra-oocyte composition.

According to another embodiment, the pharmaceutical composition further comprises a pharmaceutically acceptable carrier. According to another embodiment, the pharmaceutical composition comprises: (a) a protein selected from the group consisting of Gapd-prov, prostaglandin D2 synthetase, hematopoietic b, phosphoglucomutase 1, hypothetical protein LOC100101274, hypothetical protein LOC398635, vitellogenin (VTG)-A1, short-VTG-A1, nucleoside diphosphate kinase A1, mg:bb02e05, adenosylhomocysteinase A, and a combination thereof; and (b) an miRNA selected from the group consisting of hsa-miR-17-5p, hsa-miR-18a, hsa-miR-92a, hsa-miR-19b-1, hsa-miR-20a, mmu-miR-92a, mmu-miR-93, hsa-miR-367, hsa-miR-372, hsa-miR-373, and a combination thereof.

According to one embodiment, the administering is parenterally. According to another embodiment, the administering is selected from the group consisting of an intraperitoneal injection, a subcutaneous injection, or an intramuscular injection. According to another embodiment, the injection is an intraperitoneal injection.

According to one embodiment, the differentiated cell is selected from the group consisting of a bone marrow cell, a fibroblast cell, an adipocyte, a peripheral blood CD4+ T-lymphocyte, a buccal cell, a cancer cell, and a senescent cell. According to another embodiment, the cancer cell is selected from the group consisting of a cervical carcinoma cell, a breast adenocarcinoma cell and a melanoma cell.

According to one embodiment, the disease, disorder, condition or injury is selected from the group consisting of cancer, traumatic brain injury, traumatic alopecia, skin wrinkling and aging. According to another embodiment, the cancer is selected from the group consisting of melanoma, cervical carcinoma and breast adenocarcinoma. According to another embodiment, the cancer is melanoma.

### BRIEF DESCRIPTION OF THE DRAWINGS

FIG. 1 is a bar graph depicting the reduction in size of an induced mouse foot pad melanoma as a function of time of treatment with the pharmaceutical composition of the described invention.

FIG. 2 is a photograph of a fully-developed mouse foot pad melanoma three weeks after inoculation with B 16 cells.

FIGS. 3, 4, 5, 6, 7, and 8 are photographs of a fully-developed (40 day postinoculation) mouse foot pad melanoma after 0, 10, 20, 35, 40, and 45 days treatment respectively, with the pharmaceutical composition of the described invention.

FIG. 9 depicts photomicrographs of COX-2 immunohistological staining of sections of a mouse foot pad melanoma taken at various times of treatment with the pharmaceutical composition of the described invention.

FIG. 10 depicts photomicrographs of iNOS immunohistological staining of sections of a mouse foot pad melanoma taken at various times of treatment with the pharmaceutical composition of the described invention.

FIG. 11 is a photograph of an early-stage mouse foot pad melanoma one week postinoculation.

FIG. 12 is a photograph of the mouse foot pad melanoma of the mouse of FIG. 12 after treatment with the pharmaceutical composition of the described invention for 20 days.

FIGS. 13A to D are photographs of (A) injured mouse brains, (B) healthy mouse brains, (C) injured not treated mouse brains, and (D) injured and treated mouse brains.

FIG. 14 is a series of photographs that show the development ca. two weeks after injury and resolution of post-traumatic alopecia in a mouse after 45 days post-development treatment.

FIG. 15 is a series of photographs showing reduction in chemically-induced skin wrinkling in a mouse.

FIG. 16A is a bar graph that shows the results of mouse longevity studies. The term “Bioquantine™” is used to refer to the pharmaceutical composition of the described invention.

FIG. 16B is a bar graph presenting the results of Drosophila Melanogaster longevity studies. The term “Bioquantine™” is used to refer to the pharmaceutical composition of the described invention.

FIG. 17 is a series of photographs that show the expression of pluripotency markers by cells derived from human bone marrow stromal cells on d7 following co-electroporation with Xenopus laevis oocytes. (A)-(D) same field; (A) DAPI; (B) Oct 3/4; (C), Sox-2; (D), DAPI, Oct 3/4, and Sox-2 combined; (E)-(H) same field; (E) DAPI; (F) Oct 3/4; (G) Nanog; (H) DAPI, Oct 3/4, and Sox-2 combined; (I)-(l), same field; (1), DAPI; (J) Rex-1; (K) SSEA-1; (l) DAPI, Rex-1, and SSEA-1 combined.

FIG. 18 is a series of photographs that show the expression of pluripotency markers by cells derived from BJ cells following co-electroporation with Xenopus laevis oocytes. (A) control cells (no co-electroporation); (B)-(C) same field, dS; (B) phase contrast; (C) alkaline phosphatase; (D)-(G) same field on dS; (D) DAPI; (E) Oct 3/4; (F) Nanog; (G) DAPI, Oct 3/4, and Nanog; (H)-(I) same field, d9; (H) phase contrast, (I) TRA-1-60; (J)-(K) same field, d9; (J) phase contrast; (K) Rex-1; (L)-(M) same, field, d11; (L) phase contrast; (M) SSEA-1; (N)-(O) same field, dS; (M) phase contrast; (N) Sox-2.

FIG. 19 is a series of photographs that show the expression of pluripotency markers by cells derived from human pre-adipocytes (HPA) following co-electroporation with Xenopus oocytes. (A) duster of cells on d5 using phase contrast; (B) alkaline phosphatase; (C)-(D) same field at d5; (C) phase contrast; (D) Oct 3/4; (E)-(F) same field, d5; (E) phase contrast; (F) Nanog; (G)-(H), same field, d10; (G) phase contrast; (H) Sox-2; (I)-(J) same field, d9; (I) phase contrast; (J) TRA-1-60; (K)-(L), same field, d11; (K) phase contrast, (1) Rex-1; (M)-(N) same field, d10; (M) phase contrast, (N) SSEA-1.

FIG. 20 is a series of photographs that show the expression of neural markers by cells derived from human pre-adipocytes following culture for 8 days in conditions that promote neural progenitor differentiation by embryonic stem cells.

FIG. 21 is a series of photographs that show cells derived from human CD4+ T-lymphocytes following co-electroporation with Xenopus laevis oocytes. (A) control, no co-electroporation; (B) no co-electroporation, culture on irradiated mouse embryonic fibroblasts; (C)-(D) cell culture on d5 following coelectroporation; (E)-(F) lower part of cluster in (D); (G)-(H) alkaline phosphatase on d9.

FIG. 22 is a series of photographs that show the expression of pluripotency markers by cells derived from human CD4+ T-Lymphocytes (CD4TL) following co-electroporation with Xenopus laevis oocytes. (A)-(B), same field, d10; (A) phase contrast; (B) Oct 3/4; (C)-(D) same field, d10; (C) phase contrast; (D) Nanog; (E)-(H) same field, d5; (E) DAPI; (F) Rex-1; (G) Sox-2; (H) DAPI, Rex-1, and Sox-2; (I)-(J) same field, d9; (I) phase contrast; (J) TRA-1-60; (K)-(L), same field, d10; (K) phase contrast; (L) SSEA-1.

FIG. 23 is a series of photographs that show colonies of cells derived from human buccal mucosa cells on 6 after co-electroporation with Xenopus laevis oocytes. (A) grown on irradiated mouse embryonic fibroblast substrate; (B) grown on StemAdhere™ substrate.

FIG. 24 is a series of photographs that show the expression of human pluripotency-associated factors by cells derived from human buccal mucosa cells following co-electroporation with Xenopus laevis oocytes. (A)-(B) same field, 96 h; (A) phase contrast; (B) Oct 3/4; (C)-(D) same field, d10; (C) phase contrast; (D) Nanog; (E)-(F) same field, d10; (E) phase contrast; (F) Sox-2; (G)-(H) same field, d9, (G) phase contrast; (H) TRA-1-60; (I)-(J), same field, d11; (I) phase contrast; (J) Rex-1; (K)-(L) same field, d11; (K) phase contrast; (L) SSEA-1.

FIG. 25 is a series of photographs that show partial dedifferentiation of HeLa and MCF-7 cells following co-electroporation with Xenopus laevis oocytes. (A), HeLa cells, no co-electroporation; (B) HeLa cells grown on irradiated mouse embryonic fibroblast cells, no co-electroporation; (C) MCF-7 cells, no co-electroporation; (D) MCF-7 cells grown on irradiated mouse embryonic fibroblast cells, no co-electroporation; (E)-(H) cells derived from HeLa cells following co-electroporation with Xenopus laevis oocytes; (E)-(F), same field, d11; (E) phase contrast; (F) Oct 3/4; (G) phase contrast; (H) Oct 3/4; (I)-(L) MCF-7 cells following co-electroporation with Xenopus laevis oocytes; (G)-(H) same field, d11; (G) phase contrast; (H) Oct 3/4; (I)-(J) same field, d11; (I) phase contrast; (J) Nanog.

FIG. 26 is a table containing the spectrometry results for 93 proteins.

FIG. 27 is a bar graph that shows the distribution of hsa-miR-17-5p inside and outside Xenopus laevis oocytes before and after Bioquantine™ (BQ) activation.

FIG. 28 is a bar graph that shows the distribution of hsa-miR-18a inside and outside Xenopus laevis oocytes before and after Bioquantine™ (BQ) activation.

FIG. 29 is a bar graph that shows the distribution of hsa-miR-19a inside and outside Xenopus laevis oocytes before and after Bioquantine™ (BQ) activation.

FIG. 30 is a bar graph that shows the distribution of hsa-miR-19b inside and outside Xenopus laevis oocytes before and after Bioquantine™ (BQ) activation.

FIG. 31 is a bar graph that shows the distribution of hsa-miR-20a inside and outside Xenopus laevis oocytes before and after Bioquantine™ (BQ) activation.

FIG. 32 is a bar graph that shows the distribution of mmu-miR-92a inside and outside Xenopus laevis oocytes before and after Bioquantine™ (BQ) activation.

FIG. 33 is a bar graph that shows the distribution of mmu-miR-93 inside and outside Xenopus laevis oocytes before and after Bioquantine™ (BQ) activation.

FIG. 34 is a bar graph that shows the distribution of hsa-miR-367 inside and outside Xenopus laevis oocytes before and after Bioquantine™ (BQ) activation.

FIG. 35 is a bar graph that shows the distribution of hsa-miR-372 inside and outside Xenopus laevis oocytes before and after Bioquantine™ (BQ) activation.

FIG. 36 is a bar graph that shows the distribution of hsa-miR-373 inside and outside Xenopus laevis oocytes before and after Bioquantine™ (BQ) activation.

### DETAILED DESCRIPTION OF THE INVENTION

The described invention can be better understood from the following description of exemplary embodiments, taken in conjunction with the accompanying figures and drawings. It should be apparent to those skilled in the art that the described embodiments of the described invention provided herein are merely exemplary and illustrative and not limiting.

### DEFINITIONS

Various terms used throughout this specification shall have the definitions set out herein.

The term “adherent” as used herein refers to the act of sticking to, clinging, or staying attached.

The term “administer”, “administering” or “to administer” as used herein, refers to the giving or supplying of a medication, including in vivo administration, as well as administration directly to tissue or cells ex vivo. Generally, compositions may be administered systemically either orally, bucally, parenterally, topically, by inhalation or insufflation (i.e., through the mouth or through the nose) or rectally in dosage unit formulations containing conventional nontoxic pharmaceutically acceptable carriers, adjuvants and vehicles as desired, or may be locally administered by means such as, but not limited to, injection, implantation, grafting, topical application or parenterally.

The terms “agent” and “therapeutic agent” are used interchangeably herein to refer to a drug, molecule, composition, or other substance that provides a therapeutic effect. The term “active agent” as used herein, refers to the ingredient, component or constituent of the compositions of the described invention responsible for the intended therapeutic effect.

The term “allogeneic” as used herein refers to being genetically different although belonging to or obtained from the same species.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

The terms “residue” or “amino acid residue” or “amino acid” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

The term “attached” as used herein refers to being fastened, fixed, joined, connected, bound, adhered to or assembled with.

The term “autologous” as used herein means derived from the same organism. The term “biocompatible” as used herein refers to causing no clinically relevant tissue irritation, injury, toxic reaction, or immunological reaction to living tissue.

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention.

The term “carrier” as used herein refer to a pharmaceutically acceptable inert agent or vehicle for delivering one or more active agents to a subject, and often is referred to as “excipient.” The carrier must be of sufficiently high purity and of sufficiently low toxicity to render it suitable for administration to the subject being treated. The carrier further should maintain the stability and bioavailability of an active agent.

The term “cell” is used herein to refer to the structural and functional unit of living organisms and is the smallest unit of an organism classified as living.

The term “cellular senescence” as used herein refers to a stable and long-term loss of proliferative capacity, despite continued viability and metabolic activity. The term “replicative senescence” refers to the progressive shortening of telomeres of a given cell with replication. Senescence also can be induced in the absence of any detectable telomere loss or dysfunction, by a variety of conditions. This type of senescence has been termed premature, since it arises prior to the stage at which it is induced by telomere shortening. Premature senescence in vivo is believed to play a critical role in tumor suppression.

The term “compatible” as used herein means that the components of a composition are capable of being combined with each other in a manner such that there is no interaction that would substantially reduce the efficacy of the composition under ordinary use conditions.

The term “component” as used herein refers to a constituent part, element or ingredient.

The terms “composition” and “formulation” are used interchangeably herein to refer to a product of the described invention that comprises all active and inert ingredients. The term “active” refers to the ingredient, component or constituent of the compositions of the described invention responsible for the intended therapeutic effect. The terms “pharmaceutical formulation” or “pharmaceutical composition” as used herein refer to a formulation or composition that is employed to prevent, reduce in intensity, cure or otherwise treat a target condition or disease.

The term “condition” as used herein, refers to a variety of health states and is meant to include disorders or diseases caused by injury or any underlying mechanism or disorder.

The term “contact” and its various grammatical forms as used herein refers to a state or condition of touching or of immediate or local proximity. Contacting a composition to a target destination may occur by any means of administration known to the skilled artisan.

The term “delay”, “delaying”, “delayed” or “to delay” as used herein, refers to stopping, detaining or hindering for a time; to cause to be slower or to occur more slowly than normal.

The term “derivative” as used herein means a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.” Derivatives can include chemical modifications of the peptide, such as alkylation, acylation, carbamylation, iodination or any modification that derivatizes the peptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those peptides that contain one or more naturally occurring amino acid derivative of the twenty standard amino acids, for example, 4-hydroxyproline, 5-hydroxylysine, 3-methylhistidine, homoserine, ornithine or carboxyglutamiate, and can include amino acids that are not linked by peptide bonds. Such peptide derivatives can be incorporated during synthesis of a peptide, or a peptide can be modified by wellknown chemical modification methods (see, e.g., Glazer et al., Chemical Modification of Proteins, Selected Methods and Analytical Procedures, Elsevier Biomedical Press, New York (1975)).

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

The term “detectable response” refers to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “differential label” as used herein generally refers to a stain, dye, marker, or antibody used to characterize or contrast structures, components or proteins of a single cell or organism.

The term “differentiation” as used herein refers to the process of development with an increase in the level of organization or complexity of a cell or tissue, accompanied with a more specialized function.

The term “disease” or “disorder” as used herein, refers to an impairment of health or a condition of abnormal functioning.

The term “fluorescence” as used herein refers to the result of a three-state process that occurs in certain molecules, generally referred to as “fluorophores” or “fluorescent dyes,” when a molecule or nanostructure relaxes to its ground state after being electrically excited. Stage 1 involves the excitation of a fluorophore through the absorption of light energy; Stage 2 involves a transient excited lifetime with some loss of energy; and Stage 3 involves the return of the fluorophore to its ground state accompanied by the emission of light.

The term “functional equivalent” or “functionally equivalent” are used interchangeably herein to refer to substances, molecules, polynucleotides, proteins, peptides, or polypeptides having similar or identical effects or use.

The term “gene” as used herein refers to a region of DNA that controls a discrete hereditary characteristic, usually corresponding to a single protein or RNA. This definition includes the entire functional unit, encompassing coding DNA sequences, noncoding regulatory DNA sequences and introns.

The term “Oct4” as used herein refers to the octamer-binding transcription factor 4, also known as Oct3 and Pou5 fl, which is involved in the self-renewal or pluripotency of undifferentiated cells. Oct4 is capable of inducing a pluripotent stem cell-like state in differentiated cells. Oct4 is used as a marker for undifferentiation of a cell.

The term “Sox2” as used herein refers to the SRY (sex determining region Y)-box 2 transcription factor which is involved in maintaining self-renewal or pluripotency of undifferentiated cells. Sox2 heterodimerizes with Oct4 and is capable of inducing a pluripotent stem cell-like state in differentiated cells. Sox2 is used as a marker for undifferentiation of a cell.

The term “Klf4” as used herein refers to the Kruppel-like factor 4 transcription factor which is involved in the self-renewal or pluripotency of undifferentiated cells. Klf4 is capable of inducing a pluripotent stem cell-like state in differentiated cells. Klf4 is used as a marker for undifferentiation of a cell.

The term “Myc” as used herein refers to the transcription factor that has been linked to several cellular functions including cell-cycle regulation, proliferation, growth, differentiation and metabolism. Myc is involved in the self-renewal or pluripotency of undifferentiated cells. Myc is capable of inducing a pluripotent stem cell-like state in differentiated cells. Myc is used as a marker for undifferentiation of a cell.

The term “Nanog” as used herein refers to the transcription that is involved in maintaining self-renewal or pluripotency of undifferentiated cells. Nanog works in concert with other factors such as Oct4 and Sox2 and is capable of inducing a pluripotent stem cell-like state in differentiated cells. Nanog is used as a marker for undifferentiation of a cell.

The term “improve” (or improving) as used herein refers to bring into a more desirable or excellent condition.

As used herein, the term “inflammation” refers to a response to infection and injury in which cells involved in detoxification and repair are mobilized to the compromised site by inflammatory mediators. Inflammation often is characterized by a strong infiltration of leukocytes at the site of inflammation, particularly neutrophils (polymorphonuclear cells). These cells promote tissue damage by releasing toxic substances at the vascular wall or in uninjured tissue.

Regardless of the initiating agent, the physiologic changes accompanying acute inflammation encompass four main features: (1) vasodilation, which results in a net increase in blood flow, is one of the earliest s physical responses to acute tissue injury; (2) in response to inflammatory stimuli, endothelial cells lining the venules contract, widening the intracellular junctions to produce gaps, leading to increased vascular permeability, which permits leakage of plasma proteins and blood cells out of blood vessels; (3) inflammation often is characterized by a strong infiltration of leukocytes at the site of inflammation, particularly neutrophils (polymorphonuclear cells). These cells promote tissue damage by releasing toxic substances at the vascular wall or in uninjured tissue; and (4) fever, produced by pyrogens released from leukocytes in response to specific stimuli.

During the inflammatory process, soluble inflammatory mediators of the inflammatory response work together with cellular components in a systemic fashion in the attempt to contain and eliminate the agents causing physical distress. The terms “inflammatory” or immuno-inflammatory” as used herein with respect to mediators refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1, interleukin-4, interleukin-6, interleukin-S, tumor necrosis factor (TNF), interferon-gamma, and interleukin 12.

The term “injury” as used herein, refers to damage or harm to a structure or function of the body caused by an outside agent or force, which may be physical or chemical.

The term “isolate” and its various grammatical forms as used herein refers to placing, setting apart, or obtaining a protein, molecule, substance, nucleic acid, peptide, cell or particle, in a form essentially free from contaminants or other materials with which it is commonly associated, separate from its natural environment.

The term “labeling” as used herein refers to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The terms “marker” or “cell surface marker” are used interchangeably herein to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “microRNAs” (miRNAs) as used herein refers to a class of small non-coding RNAs (˜22 nt), which normally function as negative regulators of target mRNA expression at the posttranscriptional level by binding to the 3′UTR of target mRNAs through base pairing, resulting in target mRNAs cleavage or translation inhibition (Ambros V., Nature, 2004; 431:350-354; Bartel D. P., Cell, 2004; 116:281-297; Meister and Tuschl, Nature, 2004; 431:343-349). Increasing evidence has shown that miRNAs play critical roles in many key biological processes, such as cell growth, tissue differentiation, cell proliferation, embryonic development, cell proliferation, and apoptosis (Esquela-Kerscher and Slack, Nature Reviews Cancer, 2006; 6:259-269). As such, the mutation of miRNAs, the dysfunction of miRNA biogenesis and the dysregulation of miRNAs and their targets may result to various diseases, such as cancers (Calin and Croce, Nature Reviews Cancer, 2006; 6:857-866; Esquela-Kerscher and Slack, Nature Reviews Cancer, 2006; 6:259-269), cardiovascular disease (Latronico et al., Circ. Res, 2007; 101:1225-1236; van Rooij and Olson, J. Clin. Invest., 2007; 117:2369-2375), schizophrenia (Hansen, et al., PLos, 2007; 9:e873; Perkins et al., Genome Biology, 2007; 8:R27), renal function disorders (Williams, Cell. Mol. Life. Sci., 2008; 65:545-562), Tourette's syndrome (Esau and Monia, Advanced Drug Delivery, 2007; 59:101-114), psoriasis (Sonkoly et al., PLos, 2007: 7:e610), primary muscular disorders (Eisenberg et al., PNAS, 2007; 104:17016-17021), Fragile-X mental retardation syndrome (Fiore and Schratt, The Scientific World Journal, 2007; 7:167-177), Polycythemia vera (Bruchova et al., Experimental Hemaotlogy, 2007; 35:1657-1667), diabetes (Williams Cell. Mol. Life. Sci., 2008; 65:545-562), chronic hepatitis (Murakami et al., Oncogene, 2006; 25:2537-2545), AIDS(Hariharan et al., BBRC, 2005; 337:1214-1218), and obesity (Weiler et al., Gene Therapy, 2006; 13:496-502). The mechanisms of miRNAs implicated in diseases are very complex.

The term “modulate” as used herein means to regulate, alter, adapt, or adjust to a certain measure or proportion.

The term “multipotent” as used herein refers to a cell capable of giving rise to a limited number of cell types of a particular cell line.

The term “nucleic acid” is used herein to refer to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” is used herein to refer to a chemical compound that consists of a heterocyclic base, a sugar, and one or more phosphate groups. In the most common nucleotides, the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. Purines include adenine (A), and guanine (G); pyrimidines include cytosine (C), thymine (T), and uracil (U).

The term “parenteral” as used herein, refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersion or wetting agents and suspending agents.

The term “partition” and its various grammatical forms as used herein, refers to dividing or separating into parts or shares.

The term “peptide” is used herein to refer to two or more amino acids joined by a peptide bond.

The term “protein” is used herein to refer to a large complex molecule or polypeptide composed of amino acids. The sequence of the amino acids in the protein is determined by the sequence of the bases in the nucleic acid sequence that encodes it.

The terms “peptide”, “polypeptide” and “protein” also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. The term “pluripotent” as used herein refers to the ability to develop into multiple cells types, including all three embryonic lineages, forming the body organs, nervous system, skin, muscle and skeleton.

The term “portion” as used herein refers to a part of a whole separated from or integrated with it.

The term “prevent”, “preventing”, “prevented” or “to prevent” as used herein, refers to effectual stoppage of action or progress.

The term “progenitor cell” as used herein refers to an early descendant of a stem cell that can only differentiate, but can no longer renew itself. Progenitor cells mature into precursor cells that mature into mature (differentiated) phenotypes. Hematopoietic progenitor cells are referred to as colony-forming units (CFU) or colony-forming cells (CFC). The specific lineage of a progenitor cell is indicated by a suffix, such as, but not limited to, CFU-E (erythrocytic), CFU-F (fibroblastic), CFU-GM (granulocytic/macrophage), and CFU-GEMM (pluripotent hematopoietic progenitor).

The term “prolong”, “prolonging”, “prolonged” or “to prolong” as used herein, refers to lengthening in time, extent, scope or range.

The term “propagate” as used herein refers to reproduce, multiply, or to increase in number, amount or extent by any process.

The term “purification” as used herein refers to the process of isolating or freeing from foreign, extraneous, or objectionable elements.

The term “Reactive oxygen species” (“ROS”), such as free radicals and peroxides, as used herein refers to a class of molecules that are derived from the metabolism of oxygen and exist inherently in all aerobic organisms. The term “oxygen radicals” as used herein refers to any oxygen species that carries an unpaired electron (except free oxygen). The transfer of electrons to oxygen also may lead to the production of toxic free radical species. The best documented of these is the superoxide radical. Oxygen radicals, such as the hydroxyl radical (OH—) and the superoxide ion (O2-) are very powerful oxidizing agents that cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, may be converted by biological systems into other more reactive species, such as peroxyl (ROO—), alkoxyl (RO—) and hydroxyl (OH—) radicals.

The term “reduce”, “reducing”, “reduced” or “to reduce” as used herein, refers to a diminishing, a decrease in, an attenuation or abatement of the degree, intensity, extent, size, amount, density or number of.

The term “regeneration” or “regenerate” as used herein refers to a process of recreation, reconstitution, renewal, revival, restoration, differentiation and growth to form a tissue with characteristics that conform with a natural counterpart of the tissue.

The term “relative” as used herein refers to something having, or standing in, some significant association to something else. The term “relative frequency” as used herein refers to the rate of occurrence of something having or standing in some significant association to the rate of occurrence of something else. For example, two cell types, X cells and Y cells occupy a given location. There are 5 X cells and 5 Y cells in that location. The relative frequency of cell type X is 5/10; the relative frequency of cell type Y is 5/10 in that location. Following processing, there are 5 X cells, but only 1 Y cell in that location. The relative frequency of cell type X following processing is 5/6, and the relative frequency of cell type Y following processing is 1/6 in that location.

The term “repair” as used herein as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function. In some embodiments “repair” includes full repair and partial repair.

The term “stem cells” refers to undifferentiated cells having high proliferative potential with the ability to self-renew (make more stem cells by cell division) that can generate daughter cells that can undergo terminal differentiation into more than one distinct cell phenotype.

The term “stimulate” as used herein refers to activate, provoke, or spur. The term “stimulating agent” as used herein refers to a substance that exerts some force or effect.

The term “syndrome” as used herein, refers to a pattern of symptoms indicative of some disease or condition.

The terms “subject” and “patient” are used interchangeably herein to refer to animal species of mammalian origin that may benefit from the administration of a drug composition or method of the described invention. Examples of subjects include humans, and other animals such as horses, pigs, cattle, dogs, cats, rabbits, mice, rats and aquatic mammals.

The phrase “subject in need thereof” as used herein refers to a subject suffering from a disease, disorder, condition or injury characterized by damaged or cancerous differentiated cells that (i) will be administered a pharmaceutical composition of the described invention, (ii) is receiving a pharmaceutical composition of the described invention; or (iii) has received a pharmaceutical composition of the described invention, in order to reprogram those cells into iPSC-like cells and treat the condition, unless the context and usage of the phrase indicates otherwise.

The terms “therapeutic amount”, “therapeutically effective amount” and “amount effective” are used interchangeably herein to refer to an amount of one or more active agent(s) that is sufficient to provide the intended benefit of treatment. Dosage levels are based on a variety of factors, including the type of injury, the age, sex, weight, medical condition of the patient, the severity of the condition, the route of administration and the particular active agent employed. The dosage regimen may vary widely, but can be determined routinely by a physician using standard methods.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect also may include, directly or indirectly, the arrest reduction or elimination of the progression of a disease manifestation.

The term “treat”, “treating” or “to treat” as used herein, refers to accomplishing one or more of the following: (a) reducing the severity of a disorder; (b) limiting the development of symptoms characteristic of a disorder being treated; (c) limiting the worsening of symptoms characteristic of a disorder being treated; (d) limiting the recurrence of a disorder in patients that previously had the disorder; and (e) limiting recurrence of symptoms in patients that were previously asymptomatic for the disorder. The term “treat”, “treating” or “to treat” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms.

The term “variant” as used herein refers to a peptide sequence that varies at one or more amino acid positions with respect to the reference peptide. A variant can be a naturally-occurring variant or can be the result of spontaneous, induced, or genetically engineered mutation(s) to the nucleic acid molecule encoding the variant peptide. A variant peptide can also be a chemically synthesized variant. A skilled artisan likewise can produce polypeptide variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan.

According to one aspect, the described invention provides compositions obtained from amphibian oocytes, preferably oocytes of Xenopus laevis. One such composition is designated an intra-oocyte composition; a second such composition is designated as an extra-oocyte composition.

The compositions of the described invention comprise extracts of amphibian oocytes containing, for example, proteins (polypeptides) and microRNAs (miRNAs) (polynucleotides), in combination with a solvent.

Exemplary proteins may include, but are not limited to, a Gapd-prov protein, a prostaglandin D2 (PGD2) synthetase protein, a hematopoietic b protein, a phosphoglucomutase 1 protein, hypothetical protein LOC100101274, hypothetical protein LOC398635, a vitellogenin (VTG)-A1 protein, a short-VTG-A1 protein, a nucleoside diphosphate kinase A1 protein, mg:bb02e05 and an adenosylhomocysteinase A protein. Without limitation, for example, PGD2s function as a neuromodulator as well as a trophic factor in the central nervous system; phosphoglucomutase (PGM) is a key enzyme in glucose metabolism; vitellogenin is a female-specific glucolipoprotein yolk precursor produced by all oviparous animals, nucleoside diphosphate kinase A1 is believed to play a major role in the synthesis of nucleoside triphosphates other than ATP; and adenosylhomocysteine is a competitive inhibitor of S-adenosyl-L-methionine-dependent methyl transferase reactions, and may play a key role in the control of methylations via regulation of the intracellular concentration of adenosylhomocysteine,

Exemplary microRNAs may include, without limitation, hsa-miR-17-5p, hsa-nu/r-18a, hsa-miR-92a, hsa-miR-19b-1, hsa-miR-20a, mmu-miR-92a, mmu-miR-93, hsa-miR-367, hsa-miR-372 and hsa-miR-373.

According to some embodiments, the compositions of the present invention may further include one or more compatible active ingredients which are aimed at providing the composition with an additional pharmaceutical effect.

The compositions of the present invention may be formulated as aqueous suspensions. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid water. Among the acceptable vehicles and solvents that may be employed are water, Ringer's solution, and isotonic sodium chloride solution. In addition, sterile, fixed oils are conventionally employed as a solvent or suspending medium. For parenteral application, vehicles may consist of solutions, e.g., oily or aqueous solutions, as well as suspensions, emulsions, or dispersions. Aqueous suspensions may contain substances which increase the viscosity of the suspension and include, for example, sodium carboxymethyl cellulose, sorbitol and/or dextran. Optionally, the suspension may also contain stabilizers.

According to one embodiment, the compositions of the described invention may be prepared, for example, by a process that comprises: 1) providing a suspension of amphibian oocytes, harvested from an amphibian, in a buffered oocyte washing solution in an oocyte activation vessel; 2) applying an electroporation stimulus to the suspended oocytes in the oocyte activation vessel to produce a suspension of activated oocytes; 3) combining an aqueous energy solution with the suspension of activated oocytes; 4) incubating the combination so obtained in step 3) at an incubation temperature of 16° C. to 20° C., for an incubation time of about 2 to about 4 hours; 5) partitioning the incubated combination (for example, using a method based on density), to obtain an extra-oocyte portion (that is, the portion external to the incubated activated oocytes), and an activated oocyte portion that includes the incubated activated oocytes; and 6) separating the extra-(activated)-oocyte and the activated oocyte portions from each other.

According to another embodiment, the incubation temperature of step 4) is 16° C., 17° C., 18° C., 19° C. or 20° C.

According to one embodiment, the buffered oocyte washing solution (“OWS”) may include, but is not limited to, NaCl (at 82.5 mM), HEPES (Sigma cat.#H4034 at 5.0 mM), KCl (at 2.5 mM), MgCl₂ (at 1 mM), NaHPO₄, (at 1.0 mM), and 0.5% penicillin/streptomycin, adjusted to a pH of about 7.4. According to another embodiment, the OWS may include, but is not limited to, NaCl (at 82.5 mM), KCl (at 2.5 mM), MgCl₂ (at 1 mM), and NaHPO₄, (at 1.0 mM), adjusted to a pH of about 7.4 when used, for example, as a control in in vivo studies.

According to one embodiment, the amphibian oocytes may be suspended in buffered OWS in an electroporation vessel. It is understood that any convenient vessel can be used as the electroporation vessel provided that it can accommodate the electroporation electrodes in a manner that allows delivery of electroporation stimulus to the amphibian oocytes. Standard T25 cell culture flasks and Gene Pulser electroporation cuvettes (Bio-Rad cat. no. 165-2088) are examples of suitable electroporation vessels. According to one embodiment, the electroporation electrodes may be diagonally opposed at a separation of about 6 cm. According to one embodiment, the electroporation stimulus may be about 100 v/cm to about 200 v/cm at about 25 μF to about 75 μF applied in about 0.3 msec to about 1.5 msec pulses (time 30 constant of 0.7 to 0.9 msec.) at about 5 to 10 pulses. According to another embodiment, the electroporation stimulus may be about 125 v/cm at about 50 μF applied in about 0.3 msec to 1.5 msec pulses at about 7 pulses.

Aqueous energy solutions may be combined with the suspension of activated oocytes in order to provide, for example, chemicals or coenzymes necessary for cellular metabolism. According to one embodiment, the aqueous energy solution may comprise about 7.5 mM creatine phosphate, about 1 mM adenosine-5′-triphosphate (ATP) at pH 7.7, and about 1 mM MgCl₂. According to another embodiment, the energy solution may be a 1:100 aqueous dilution of creatine phosphate, ATP, and MgCl₂.

According to one embodiment, the combination of the aqueous energy solution with the suspension of activated oocytes may be incubated at an incubation temperature of about 16° C. to about 20° C. for an incubation time of about 1 to 4 hours. According to another embodiment, the incubation temperature may be about 16° C. According to another embodiment, the incubation temperature may be about 17° C. According to another embodiment, the incubation temperature may be about 18° C. According to another embodiment, the incubation temperature may be about 19° C. According to another embodiment, the incubation temperature may be about 20° C. According to another embodiment, the incubation time may be at least about 2 hours but not more than about 4 hours. According to another embodiment, the incubation time may be about 3 hours.

According to one embodiment, the incubated combination that includes activated oocytes may be partitioned to obtain an extra-oocyte portion and an activated oocyte portion that contains activated, incubated amphibian oocytes. According to one embodiment, partitioning may be accomplished by methods based on differences in density, for example, by centrifugation. According to one embodiment, the partitioning includes, for example, conditions that do not rupture the activated incubated oocytes. According to another embodiment, the conditions include, but are not limited to, centrifugation at a force not exceeding about 52×g.

According to one embodiment, the extra-oocyte portion may be separated from the activated oocyte portion.

According to one embodiment, the extra-oocyte composition may be obtained, for example, by filtration. According to one embodiment, the extra-oocyte portion may be filtered through a fine filter. Filters can be obtained from Sigma, Fisher Scientific, or other commercial sources familiar to those skilled in the art. According to another embodiment, the fine filter may have a pore size from about 0.01μ to 1μ. According to another embodiment, the fine filter may have a pore size of about 0.02μ. According to another embodiment, the fine filter may have a pore size of about 0.03μ. According to another embodiment, the fine filter may have a pore size of about 0.04μ. According to another embodiment, the fine filter may have a pore size of about 0.05μ. According to another embodiment, the fine filter may have a pore size of about 0.06μ. According to another embodiment, the fine filter may have a pore size of about 0.07μ. According to another embodiment, the fine filter may have a pore size of about 0.08μ. According to another embodiment, the fine filter may have a pore size of about 0.09μ. According to another embodiment, the fine filter may have a pore size of about 0.1μ. According to another embodiment, the fine filter may have a pore size of about 0.2μ. According to another embodiment, the fine filter may have a pore size of about 0.2μ. According to another embodiment, the fine filter may have a pore size of about 0.3μ. According to another embodiment, the fine filter may have a pore size of about 0.4μ. According to another embodiment, the fine filter may have a pore size of about 0.5μ. According to another embodiment, the fine filter may have a pore size of about 0.6μ. According to another embodiment, the fine filter may have a pore size of about 0.7μ. According to another embodiment, the fine filter may have a pore size of about 0.8μ. According to another embodiment, the fine filter may have a pore size of about 0.9μ. According to another embodiment, the fine filter may have a pore size of about 1.0μ.

According to another embodiment, the extra-oocyte portion may be combined either before or after filtration with either a protease inhibitor (e.g. Sigma cat# P8340) or a Rnase inhibitor (e.g. SUPERase In Rnase, Applied Biosystems cat# AM2694) to obtain the extra-oocyte composition of the described invention. According to another embodiment, the extra-oocyte portion may be combined either before or after filtration with both a protease inhibitor (e.g. Sigma cat# P8340) and an RNase inhibitor (e.g. SUPERase In RNase, Applied Biosystems cat# AM2694) to obtain the extra-oocyte composition of the described invention. According to another embodiment, the extra-oocyte portion is maintained at a temperature of about 2° C. to 8° C. According to another embodiment, the extra-oocyte portion is maintained at a temperature of about 4° C.

According to one embodiment, the intra-oocyte composition may be obtained from the activated oocyte portion by methods including, but not limited to, centrifugation. For example, the activated oocyte portion may be suspended in OWS and centrifuged under conditions that do not rupture the activated oocytes, but that provide a “pellet” of activated oocytes. After centrifugation, residual OWS then may be carefully removed from the pellet of activated oocytes by techniques well-known to those skilled in the art. The pellet of activated oocytes from which OWS has been removed may be centrifuged, for example, at 10,000 rpm at a temperature below 20° C., to rupture the activated oocytes and provide three fractions: a light fraction, a heavy fraction, and a fraction of intermediate density. The light fraction, which may be two-phased, includes, for example, yolk proteins. The heavy fraction includes, for example, cell membranes and yolk particles. The fraction of intermediate density (i.e., the cytoplasmic fraction), will become the intra-oocyte composition of the described invention. The cytoplasmic fraction is separated from the light and heavy fractions by techniques well-known to those skilled in the art. The cytoplasmic fraction may be combined with a protease inhibitor (e.g. Sigma cat# P8340) and an RNase inhibitor (e.g. SUPERase In RNase, Applied Biosystems cat# AM2694). The cytoplasmic fraction containing the inhibitors may be cooled at about 4° C. for up to about one-half hour. The intra-oocyte composition of the described invention is obtained by filtering the cooled cytoplasmic fraction containing the inhibitors through a fine filter, e.g., one having a pore size of about 0.2μ.

According to another aspect, the described invention provides a pharmaceutical composition comprising a therapeutic amount of the oocyte compositions of the described invention, which is effective to reprogram damaged or cancerous differentiated cells into iPSC-like cells that achieve regeneration, replacement, repair and/or rejuvenation of the damaged or cancerous differentiated cells and thereby treat the disease, condition, injury or disorder being characterized by the damaged or cancerous differentiated cells. Nonlimiting examples of such diseases, conditions, injuries or disorders include melanoma, traumatic brain injury, post-traumatic alopecia, and skin wrinkling.

The pharmaceutical composition can be formulated by mixing equal volumes of the extra-oocyte composition and the intra-oocyte compositions of the described invention. The pharmaceutical compositions comprise proteins and microRNAs. According to some embodiments, the protein component can include protease-resistant forms of aGapd-prov protein, a prostaglandin D2 synthase protein, a hematopoietic b protein, a phosphoglucomutase 1 protein, hypothetical protein LOC 100101274, hypothetical protein LOC398635, a vitellogenin-A1 protein, a short-VTG-A1 protein, a nucleoside diphosphate kinase A1 protein, a mg:bb02e05 protein, an adenosylhomocysteinase A protein and combinations thereof. According to some embodiments, the microRNA component can include, for example, hsa-miR-17-5p, hsa-miR-18a, hsa-miR-92a, hsa-miR-19b-1, hsa-miR-20a, mmu-miR-92a, mmu-miR-93, hsa-miR-367, hsa-miR-372 and hsa-miR-373. The pharmaceutical composition of the described invention can comprise about 5 mg/mL solid oocyte material, as determined by lyophilization experiments.

According to some embodiments, the pharmaceutical compositions of the present invention may be formulated with an excipient or carrier. The carrier can be inert, or it can possess pharmaceutical benefits. The carrier can be liquid or solid and is selected with the planned manner of administration in mind to provide for the desired bulk, consistency, etc., when combined with an active and the other components of a given composition. The term “pharmaceutically acceptable carrier” as used herein refers to any substantially non-toxic carrier conventionally useful for administration of pharmaceuticals in which the active component will remain stable and bioavailable. In some embodiments, the pharmaceutically acceptable carrier of the compositions of the present invention include a release agent such as a sustained release or delayed release carrier. In such embodiments, the carrier can be any material capable of sustained or delayed release of the actives to provide a more efficient administration, resulting in less frequent and/or decreased dosage of the active ingredient, ease of handling, and extended or delayed effects. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

Additional pharmaceutical compositions of the present invention can be readily prepared using technology which is known in the art such as described in Remington's Pharmaceutical Sciences, 18th or 19th editions, published by the Mack Publishing Company of Easton, Pa., which is incorporated herein by reference.

The pharmaceutical composition may be constituted into any form suitable for the mode of administration selected. Exemplary routes of administration include, but are not limited to, parenteral (including subcutaneous), oral, inhalation, insufflation, topical, buccal and rectal. Compositions suitable for parenteral administration include sterile solutions, emulsions and suspensions. Oral administration include solid forms, such as pills, capsules, granules, tablets, and powders, and liquid forms, such as solutions, syrups, elixirs, and suspensions. Compositions suitable for inhalation and insufflation may take the form of an aerosolized solution. Compositions suitable for topical administration include creams, ointments and dermal patches. Compositions suitable for buccal administration may take the form of tablets or lozenges. Compositions suitable for rectal administration may take the form of suppositories. Formulations for administration may be provided using any formulation known in the art and appropriate for the route of administration. Such formulations may be as provided using the guidance of such resources as REMINGTON'S PHARMACEUTICAL SCIENCES, 18th ed., Mack Publishing Co., Easton, Pa. 1990.

Use to Treat/Inhibit Progression of Melanoma

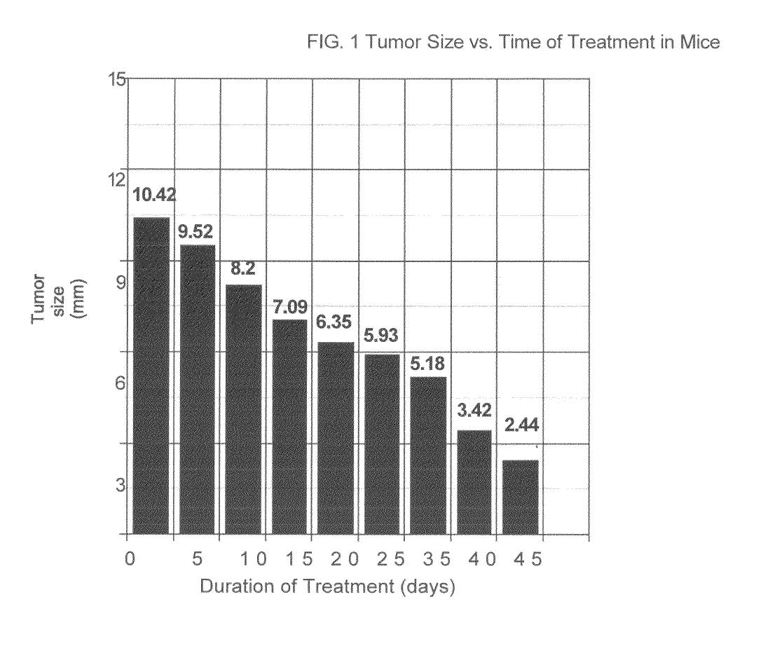

According to one embodiment, the described invention provides a method for treating or inhibiting the progression of melanoma in a mammal. The method includes the steps of (a) preparing the extra-oocyte composition and the intra-oocyte composition by acquiring the oocytes; activating the oocytes; incubating the oocytes, partitioning the incubated combination; separating the extra-oocyte portion from the activated oocyte portion as described above; (b) formulating the pharmaceutical composition; and (c) administering a therapeutically-effective amount of the pharmaceutical composition of the described invention to a mammal suffering from melanoma. According to one embodiment, the administration may be by injection or i.v. drip. According to another embodiment, the injection may be, for example, an intraperitoneal injection, a subcutaneous injection, or an intramuscular injection. According to another embodiment, the injection may be an intraperitoneal injection. According to another embodiment, the efficacy of treating or inhibiting the progression of melanoma in a mammal may be demonstrated by, for example, a decrease in tumor mass with time of treatment relative to controls. See, e.g., FIG. 1.

It is understood that the therapeutically-effective amount of the pharmaceutical composition will depend on the type of injury, the age, weight, sex, medical condition of the patient, the severity of the condition, the route of administration, and the particular active agent employed. Thus the dosage regimen may vary widely, but can be determined routinely by a physician using standard methods. According to one embodiment, the therapeutically-effective amount may vary about a mean of about 25 mg/kg body weight.

It is understood that, unlike treatment of acute conditions such as bacterial infections, successful systemic treatment of melanoma and other cancers may require more than a single, short-term course of treatment. Accordingly, according to one embodiment, treatment of melanoma with the pharmaceutical composition of the described invention may involve multiple administrations over a period of time. Those skilled in the art will know to adjust the frequency and duration of treatment, and also the amounts administered, based on patient tolerance and clinical evaluation of the regression of the disease in a particular patient.

According to one embodiment, the described invention can replace or supplement methods of treating melanoma according to the then current standard of care, such as surgery, radiation and chemotherapy.

Use to treat TBI

According to another embodiment, the described invention provides a method for treating traumatic brain injury (“TBI”) in a mammal. The method includes the steps of (a) preparing the extra-oocyte composition and the intra-oocyte composition by acquiring the oocytes; activating the oocytes; incubating the oocytes, partitioning the incubated combination; separating the extra-oocyte portion from the activated oocyte portion as described above; (b) formulating the pharmaceutical composition; and (c) administering a therapeutically-effective amount of the pharmaceutical composition of the described invention to a mammal suffering from TBI. According to one embodiment, the efficacy of treatment may be demonstrated by rate of restoration of TBI-induced memory loss, visual inspection of changes in injured brains, and resolution of TBI-induced P-amyloid plaques, relative to controls. See working example 6. According to another embodiment, administration may be by injection or i.v. drip. According to another embodiment, the injection may be, for example, an intraperitoneal injection, a subcutaneous injection, or an intramuscular injection. According to another embodiment, the injection may be an intraperitoneal injection.

It is understood that the therapeutically-effective amount of the pharmaceutical composition will depend on the type of injury, the age, weight, sex, medical condition of the patient, the severity of the condition, the route of administration, and the particular active agent employed. Thus the dosage regimen may vary widely, but can be determined routinely by a physician using standard methods. According to one embodiment, the therapeutically-effective amount may vary about a mean of about 25 mg/kg body weight.

It is understood that the disabilities resulting from TBI are variable and recovery is highly individualized. Those skilled in the art will know to adjust the frequency and duration of treatment, and also the amounts administered, based on the extent of the injury, patient tolerance and clinical evaluation of the regression of the disease or the progress of recovery in a particular patient. According to one embodiment, treatment of the TBI with the pharmaceutical composition of the described invention may involve multiple administrations over a period of time.

Use to Treat Trauma-Induced Alopecia

According to one embodiment, the described invention provides a method for treating trauma-induced alopecia. The method includes the steps of (a) preparing the extra-oocyte composition and the intra-oocyte composition by acquiring the oocytes; activating the oocytes; incubating the oocytes, partitioning the incubated combination; separating the extra-oocyte portion from the activated oocyte portion as described above; (b) formulating the pharmaceutical composition; and (c) administering to a mammal suffering from trauma-induced alopecia. According to one embodiment, administration may be by injection or i.v. drip. According to another embodiment, the injection may be, for example, an intraperitoneal injection, a subcutaneous injection, or an intramuscular injection. According to another embodiment, the injection may be an intraperitoneal injection.

It is understood that the therapeutically-effective amount of the pharmaceutical composition will depend on the type of injury, the age, weight, sex, medical condition of the patient, the severity of the condition, the route of administration, and the particular active agent employed. Thus the dosage regimen may vary widely, but can be determined routinely by a physician using standard methods. According to one embodiment, the therapeutically-effective amount may vary about a mean of about 25 mg/kg body weight.

It is understood that the disabilities resulting from trauma-induced alopecia are variable and recovery is highly individualized. Those skilled in the art will know to adjust the frequency and duration of treatment, and also the amounts administered, based on the extent of the injury, patient tolerance and clinical evaluation of the regression of the disease or the progress of recovery in a particular patient. According to one embodiment, treatment of melanoma with the pharmaceutical composition of the described invention may involve multiple administrations over a period of time.

Use to Treat Aging Skin

According to one embodiment, the described invention provides a method of treating cellular senescence in a mammal as exemplified by aging skin (skin-wrinkling) The method includes, but is not limited to, the steps of (a) preparing the extra-oocyte composition and the intra-oocyte composition by acquiring the oocytes; activating the oocytes; incubating the oocytes, partitioning the incubated combination; separating the extra-oocyte portion from the activated oocyte portion as described above; (b) formulating the pharmaceutical composition; and (c) administering a therapeutically-effective amount of the pharmaceutical composition of the described invention. According to one embodiment, administration may be by injection or i.v. drip. According to another embodiment, the injection may be, for example, an intraperitoneal injection, a subcutaneous injection, or an intramuscular injection. According to another embodiment, the injection may be an intraperitoneal injection.

It is understood that the therapeutically-effective amount of the pharmaceutical composition will depend on the type of injury, the age, weight, sex, medical condition of the patient, the severity of the condition, the route of administration, and the particular active agent employed. Thus the dosage regimen may vary widely, but can be determined routinely by a physician using standard methods. According to one embodiment, the therapeutically-effective amount may vary about a mean of about 25 mg/kg body weight.

It is understood that the skin-wrinkling in a patient is variable and regression is highly individualized. Those skilled in the art will know to adjust the frequency and duration of treatment, and also the amounts administered, based on the extent of wrinkling, patient tolerance and clinical evaluation of the regression of wrinkling in a particular patient. According to one embodiment, treatment of melanoma with the pharmaceutical composition of the described invention may involve multiple administrations over a period of time.

Use to Prolong Life Expectancy

According to another embodiment, the described invention provides a method for increasing the life expectancy of a mammal or invertebrate, relative to respective control cohorts by effecting reprogramming of senescent and/or apoptotic cells. The method includes the steps of (a) preparing the extra-oocyte composition and the intra-oocyte composition by acquiring the oocytes; activating the oocytes; incubating the oocytes, partitioning the incubated combination; separating the extra-oocyte portion from the activated oocyte portion as described above; (b) formulating the pharmaceutical composition; and (c) administering the pharmaceutical composition of the described invention. According to one embodiment, administration may be by injection or i.v. drip. According to another embodiment, the injection may be, for example, an intraperitoneal injection, a subcutaneous injection, or an intramuscular injection. According to one embodiment the administering may be intraperitoneally to a mammal or in the food of an invertebrate.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “an”, and “the” include plural references unless the context clearly dictates otherwise. All technical and scientific terms used herein have the same meaning.

The publications discussed herein are provided solely for their disclosure prior to the filing date of the present application and each is incorporated by reference in its entirety. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Preparation and Maintenance of Xenopus laevis oocytes

In this study, oocytes in the final stage of maturity were collected from Xenopus laevis.

South African clawed, egg-bearing frogs (Xenopus laevis, NASCO cat# LM00531, Fort Atkinson Wis., USA) were adapted to the new environment for two weeks at about 18° C. using a 12/12-hour light/dark cycle, and were kept in carbon-filtered water supplemented with 13.3 g/gallon sodium chloride. Animals were fed frog brittle (NASCO cat# SA02764LM). Water in containers was replaced on a daily basis. Eggs (oocytes) were then surgically harvested.

Prior to surgery, frogs were anesthetized in a plastic beaker containing 1 L of 0.2% tricane solution (Sigma cat# A5040) for up to 20 min, then, placed on a dissecting pan filled with ice. A small incision (0.5 cm) was made through the skin layer and then the muscle layer. The bags of the ovaries were surgically removed and placed into buffered oocyte washing solution (OWS) containing 82.5 mM NaCl (Sigma cat# S3014), 5.0 mM HEPES (Sigma cat# H4034), 2.5 mM KCl (Sigma cat# P5405), 1 mM MgCl₂ (Sigma cat# M0250), 1.0 mM Na₂HPO, (Sigma cat.#S3264), and 0.5% penicillin/streptomycin. The pH was adjusted to 7.4.

Bags containing oocytes were disrupted with fine forceps and rinsed multiple times with OWS. After a final rinse, any remaining follicular cell layers were digested by placing the oocytes into a 0.2% collagenase type II solution (Worthington Biochemical Corporation cat#LS004176, Lakewood, N.J.) for one hour or more at room temperature. Defolliculated oocytes were rinsed in OWS and then placed for overnight incubation in a fresh holding buffer (HB) containing 5 mM NaCl, 5.0 mM HEPES, 2.5 mM KCl, 1 mM MgCl₂, 1.0 mM Na₂HP04, 0.5% penicillin/streptomycin, 1.0 mM CaCl₂ (Sigma cat#223506), 2.5 mM pyruvate, and 5% heat-inactivated horse serum (Sigma cat# H1138) titrated to pH 7.4.

Recovered oocytes in the final stage of maturity were collected in sterile 6-well cell culture clusters (Costar cat#3516) prefilled with HB and then incubated at 17° C. in a low-temperature incubator for 24 hours before they were collected for electroporation and preparation of extra-oocyte and intra-oocyte portions.

### Example 2

### Preparation of Intra-Oocyte and Extra-Oocyte Compositions

In this study, oocytes collected in Example 1 were used to prepared intra-oocyte and extra-oocyte compositions. Intra-oocyte and extra-oocyte compositions were separated in order to maintain the two different phases and to interrupt the timed (3 h) process of semiochemical emission.

Defolliculated Xenopus oocytes obtained by the procedure of Example 1 were rinsed 5 times in HEPES free and penicillin/streptomycin free OWS. Approximately 1,000 oocytes were transferred to each of several sterile T25 cell culture flasks containing 10 ml of fresh OWS and equipped with two electrodes positioned diagonally at a separation of 6 cm. Oocytes were electroporated using the following parameters: 750 volts (125 v/cm), 50 μF, 7 pulses, with time constant at 0.7-0.9 msec.

After electroporation, 100 μl of each of three stock energy solutions (7.7 mM creatine 25 phosphate, 1 mM ATP at pH 7.7, and 1 mM MgCl₂) were added to each flask containing electroporated oocytes. Flasks were then placed in a low-temperature incubator on an orbital shaker at 17° C. and rotated for 3 hr.

Following incubation, the electroporated (i.e., activated) oocytes were transferred to 50 ml conical tubes and partitioned by centrifuging at 52×g for 7 min. Approximately 10 mL of supernatant extra-oocyte portion was removed from each tube, combined with 500 μl of SUPERase-In RNase inhibitor (Applied Biosystems cat# AM2694) at a final concentration of 1 U/μl and 100 μl (1:100 dilution) of protease inhibitor cocktail (Sigma cat# P8340). The combinations were kept on ice during the procedure. The chilled combinations were filtered cold through a pre-chilled 115 ml, 0.2 μm filter unit (Nalgene cat#121-0020, Rochester, N.Y.) to obtain extra-oocyte composition.

In order to obtain the intra-oocyte composition, pellets of activated oocytes from each tube containing only activated oocytes (i.e. containing only activated oocyte portion) were gently suspended in OWS (by swirling) and then transferred into 12 ml polypropylene adapter tubes (Sarstedt). Tubes were centrifuged in a clinical centrifuge at 150×g for 30 seconds, then at 700×g for 30 seconds at 16° C. All excess buffered OWS was removed from the top of the packed oocytes in order to obtain a concentrated cytoplasm.

Tubes with oocytes were transferred onto a high speed (HS) refrigerated centrifuge and centrifuged at 10,000 rpm for 15 minutes at 16° C. to rupture the oocytes. After HS centrifugation, tubes were placed in ice. HS centrifugation produced three fractions: a light fraction (a yellow lipid layer at the top of the tube); a heavy fraction at the bottom of the tube (heavy membranes and yolk particles); and a fraction of intermediate density (the cytoplasmic layer) between the light and heavy fractions.

The sides of the tubes were wiped with a tissue before piercing with a 20G needle at the bottom of each cytoplasmic fraction. The cytoplasmic fraction contains essential components of the intra-oocyte composition and was carefully removed by syringe. The cytoplasmic fractions were chilled on ice and combined with 500 μl of SUPERase-In RNase inhibitor (Applied Biosystems cat# AM2694) at a final concentration of 1 U/μ1 and 100 μl (1:100 dilution) of protease inhibitor cocktail (Sigma cat# P8340).

The combination was incubated on ice for 20 min., then filtered in pre-cooled 115 mL, 0.2 μm filter units (Nalgene cat#121-0020, Rochester, N.Y.) to obtain the intra-oocyte composition.

### Example 3

### Formulation and Analysis of Pharmaceutical Composition

In this study, the intra-oocyte composition obtained in Example 2 was formulated for intraperitoneal and subcutaneous injection.

The collected intra-Oocyte and extra-Oocyte compositions from Example 2 were combined in equal volumes (5 ml+5 ml) into sterile 10 mL glass serum vials. All vials containing the pharmaceutical composition were subsequently store in the dark at 4° C.

The concentration of solids in the pharmaceutical composition was determined by lyophilizing measured volumes of the pharmaceutical composition in pre-weight lyophilization vials. The concentration of solids was found to be 5±0.5 mg/mL.

The pharmaceutical composition obtained was tested for bacteria using a Gram Staining Kit (Fluka cat#77730) according to manufacturer's protocol. The pharmaceutical composition was also tested for mycoplasma contamination using a PCR-based Universal Mycoplasma Detection Kit (ATCC cat#30-1012K) according to manufacturer's protocol. Negative results were obtained from both the Gram Staining Kit and Universal Mycoplasma Detection Kit. Therefore, the pharmaceutical composition was deemed safe for intraperitoneal and subcutaneous injection.

### Example 4

### Treatment of Melanoma in a Mouse Foot Pad Model

Melanoma is a tumor derived from genetically altered epidermal melanocytes that arises because of complex interactions between genetic and environmental factors. The etiological pathogenesis of human melanoma is attributed to the combination of genetic predisposition and exposure to ultraviolet radiation (UVR). The transformation of epidermal melanocytes, and the progression from localized tumor to metastatic disease, occurs in a stepwise process resulting from the differential expression of genes. Four critical molecular phases in the development and progression of melanoma have been identified: (1) onset of genetic instability, (2) enhanced and inappropriate cellular proliferation, (3) acquisition of invasive and metastatic traits, and (4) promotion of tumor angiogenesis (See, e.g., Sulaimon S. S, and Kitchell B. E., J. Vet. Intern. Med., 2003; 17:760-772).

### (1) Onset of Genetic Instability

Disrupting the genetic integrity of the melanocyte is a critical event in the development of melanoma. Factors known to alter the melanocyte genome, resulting in a genetically unstable melanocyte, include: infidelity of DNA replication; defects in DNA repair; generation of reactive oxygen species (ROS); and spontaneous deamination of pyrimidines. Cytogenetic evaluations of human melanomas have shown that important chromosomal aberrations occur on chromosomes 1, 6, 7, and 9. One frequently studied gene shown to play a role in the dysregulated proliferation of melanoma cells is the cyclin-dependent kinase inhibitor 1A (CDKN1A) gene. This gene is located on chromosome 6 and is rearranged in human melanoma. CDKN1A is a potent inhibitor of cyclin-dependent kinases (CDKs). CDKs are necessary to regulate transitions between different phases of the cell cycle. In melanoma cells, CDKN1A control of CDKs (e.g., CDK2) is lost, resulting in dysregulated proliferation and an invasive phenotype (See, e.g., Sulaimon S. S, and Kitchell B. E., J. Vet. Intern. Med., 2003; 17:760-772).

### (2) Enhanced and Inappropriate Cellular Proliferation

Deregulated proliferation of melanocytes is facilitated primary by UVR. UVR stimulates, among others, the generation of reactive oxygen species (ROS). The ROS family includes superoxide (O₂), hydrogen peroxide (H₂O₂), hydroxyl radical (OH), hypochlorite (HOCl), nitric oxide (NO) and sometimes singlet oxygen. Members of the ROS family are highly reactive and mediate the degradation of membranes, DNA strand breaks, chromosomal abnormalities, oxidative base modifications and enzyme deactivation. The damage caused by ROS leads to cellular dysfunction, cellular transformation and/or cell death (See, e.g., Sulaimon S. S, and Kitchell B. E., J. Vet. Intern. Med., 2003; 17:760-772).

### (3) Acquisition of Invasive and Metastatic Traits

In order for melanoma cells to metastasize, the cells must first release themselves from intercellular adhesive bonds. This is accomplished by secreting proteolytic enzymes such as matrix metalloproteinases. Once the cells leave the normal cellular microenvironment and migrate through the connective tissue matrix, they gain access to blood and lymphatic vessels. Once in the circulation, these metastatic melanoma (MM) cells must be able to survive the mechanical stress of the blood vasculature. Survival of MM cells in the circulation is accomplished by preventing apoptosis. MM cells have developed several mechanisms to escape death by apoptosis. For example, UVR induces the expression of COX-2 in MM cells. COX-2 synthesizes prostaglandin E2 (PGE2) which in turn stimulates overexpression of Bc1-2 protein. Bc1-2 protein acts to bind and inhibit the pro-apoptotic proteins BCL-2 associated x protein (Bax) and BCL-2 antagonist killer 1 protein (BAK) which blocks apoptosis (See, Chipuk J. E. and Green D. R., Trends in Cell Biology, 2008; 18(4):157-164 and Fosslien E., Ann. Clin. Lab. Sci., 2000; 30(1):3-21). In addition, MM cells are known to express inducible nitric oxide synthase (iNOS). iNOS catalyzes the production of the inflammatory mediator nitric oxide (NO). NO has been shown to protect MM cells from apoptosis by nitrosylating and inactivating caspase 9, an essential protein in the apoptotic pathway (See, Ellerhorst J. A. et al., Oncol. Rep., 2010; 23(4):901-907; Salvucci O. et al., Cancer Res., 2001; 61:318-326 and Torok N. J. et al., Cancer Res., 2002; 62:1648-1653).

Inflammation also has been implicated in the invasiveness of melanoma cells and their ability to metastasize. Inflammation generally is a protective response elicited by injury or destruction of tissues, which serves to destroy, dilute, or wall off both the injurious agent and the injured tissue. The classic signs of inflammation are heat, redness, swelling, pain, and loss of function. These are manifestations of the physiologic changes that occur during the inflammatory process. The three major components of this process are (1) changes in the caliber of blood vessels and the rate of blood flow through them (hemodynamic changes); (2) increased capillary permeability; and (3) leukocytic exudation (See, e.g., Paul, Fundamentals of Immunology).

Tumor cells, such as melanoma cells, are capable of producing various cytokines and chemokines that attract leukocytes. Leukocytes are capable of producing an assorted array of cytokines, cytotoxic mediators (e.g., NO), membrane perforating agents and soluble mediators of cell killing (e.g., TNF-a, interleukins and interferons) (See, Coussens L. M. and Werb Z., Nature, 2002; 420:859-867). Tumor cells, such as melanoma cells, not only take advantage of the trophic factors made by inflammatory cells, but may also use the same adhesion molecules, chemokines and receptors to aid in migration and homing during distant metastatic spread. Evidence suggests that mechanisms used for homing of leukocytes may be appropriated for the dissemination of tumors via the bloodstream and lymphatics. (See, Coussens L. M. and Werb Z., Nature, 2002; 420:859-867). For example, Selectins are adhesion receptors that normally recognize certain vascular mucin-type glycoproteins bearing the carbohydrate structure sialyl-Lewis X and facilitate leukocyte rolling along the blood vessels. Metastatic progression of many epithelial carcinomas, including melanoma cells, correlates with tumor production of mucins containing sialyl-Lewis X (See, Coussens L. M. and Werb Z., Nature, 2002; 420:859-867).

### (4) Promotion of Tumor Angiogenesis

Like all tumors, MM cells are dependent on adequate vasculature. Interactions between stromal and melanomal cells play a critical role in the development of neoangiogenesis in MM. MM cell hypoxic signals induce the expression and release of angiogenic factors (vascular endothelial growth factor [VEGF], beta FGF [b-FGF], IL-8, transforming growth factors alpha and beta [TGF-a and TGF-b], and endothelial cell derived growth factor) and a concurrent decrease in the production of the angiogenic inhibitors thrombospondin, interferon a and b (IFN-a and IFN-b), and angiostatin. Angiogenic factors stimulate the growth of new blood vessels and allow the transport of tumor cells into systemic circulation. The angiogenic molecules VEGF and IL-8 appear to play the most important role in the neoangiogenesis of MM (See, e.g., Sulaimon S. S, and Kitchell B. E., J. Vet. Intern. Med., 2003; 17:760-772).

In this study, mice were used to test whether the pharmaceutical composition of Example 3 could reduce the size of a melanoma tumor.

Mice: Three to four week old, immunocompetent mice were purchased from Pet World Warehouse (Madison, Wis.). Males were separated from females and distributed 5 mice per cage. The experimental and control groups consisted of 10 mice each. Animals were kept on a normal day-night cycle (L:D 12:12 h) and fed commercially available food consisting of dried fruits, grains and raw unsalted mixed nuts. Mice were adapted to the environment to the age of 8 weeks.

Induction of Melanoma: B16 melanoma cells were obtained from American Type Culture Collection (ATCC, cat# CRL-6323, Manassas, Va.) and were received frozen in vials. Cells were thawed, washed, and grown at 37° C. and 5% C0₂ in non-pyrogenic, sterile, ventilated (0.2μ), 25 cm² cell culture flasks (T25; Coming cat#3056, Corning, N.Y., USA) containing 5 mL of high glucose DMEM (Millipore cat# SLM-220M) supplemented with 10% fetal bovine serum (FBS; ATCC cat#30-2020), 1 mM sodium pyruvate (Sigma cat# P2256), 0.1 mM non-essential amino acids (NEAA; Gibco cat#11140), and 1% penicillin (50 U/mL)/streptomycin (50˜g/mL) solution (1% penicillin/streptomycin; GIBCO cat#15140).

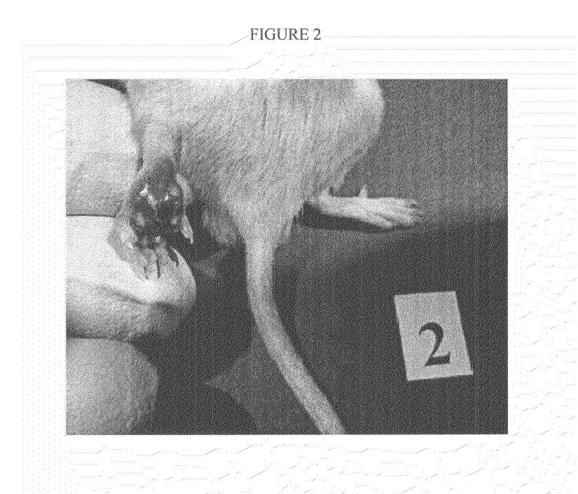

B16 cells were detached by trypsinization at confluency, washed, counted, and diluted in phosphate buffered saline solution (pH 7.4) to a concentration of 10⁶ cells/mL. Each of 10 experimental mice were inoculated with 100 μl of the solution (containing around 10⁵ melanoma cells) by subcutaneous injection into either the left or right food pad of each of the 10 mice in the experimental group. 100 μl of high glucose DMEM were subcutaneously injected into either the right or left foot pad of each of the 10 mice in the control group. Palpable primary tumors were detected in all mice in the experimental group between 12 and 14 days after injection with B16 cells. An example of a fully-developed foot pad melanoma in experimental mouse #2 three weeks after injection is shown in FIG. 2.

Treatment of Melanoma:

Beginning at post-tumor induction day 24 to day 28 (after melanoma was fully developed), each mouse in the experimental group received daily injections of the pharmaceutical composition of Example 3 for a period of 25 to up to 45 days, depending on the rate of tumor shrinkage in the individual animal.

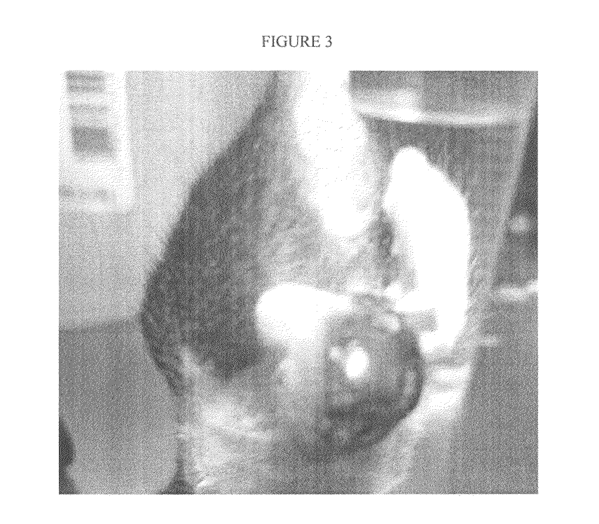

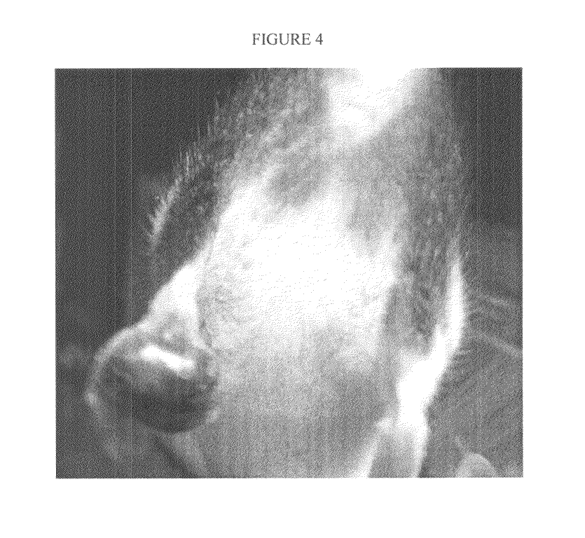

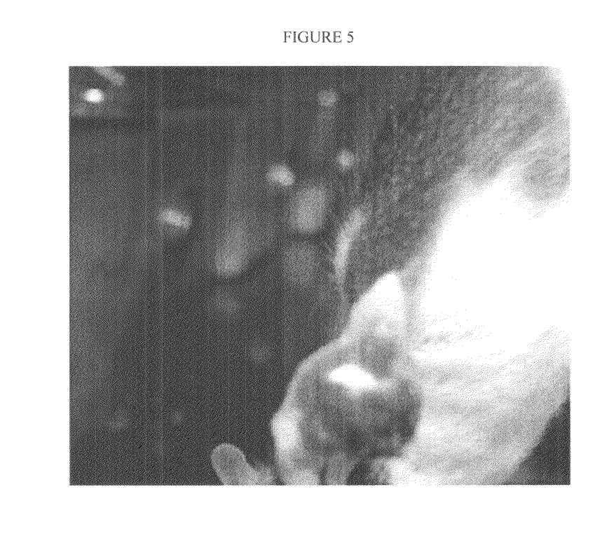

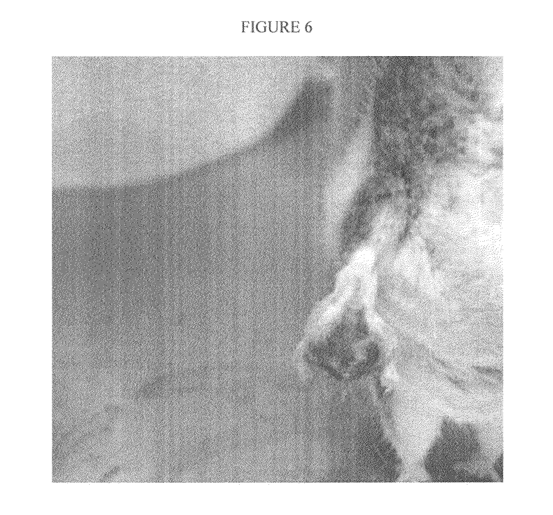

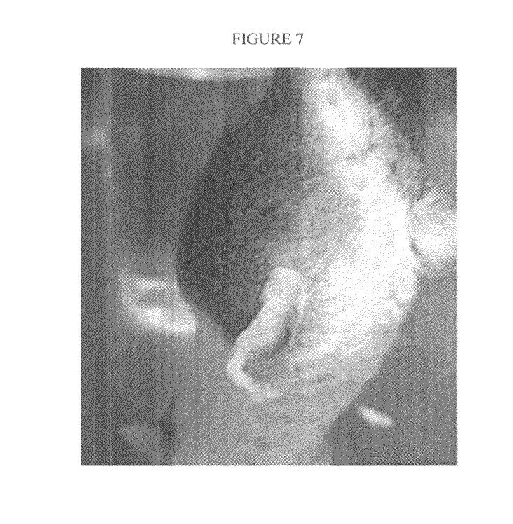

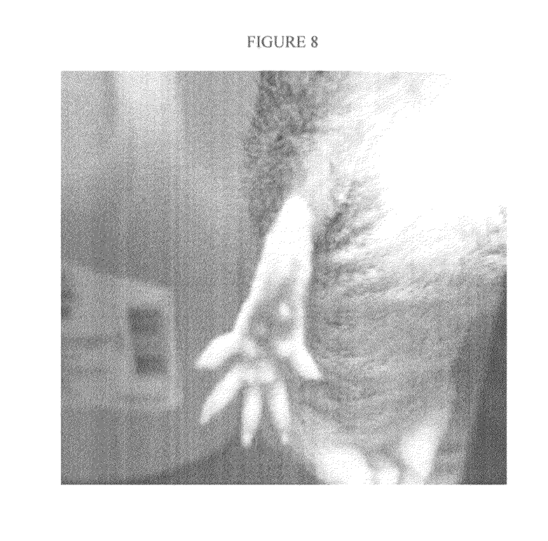

Tumor shrinkage was observed in all mice in the experimental group. The global average tumor size as a function of days of treatment for all mice is depicted in the bar chart of FIG. 1. A photographic record of tumor shrinkage as a function of days of treatment for an experimental mouse is provided by FIGS. 3 to 8 as shown in Table 1 which follows:

TABLE 1

| Figure | Days of Treatment |
|---|---|
| 3 | 0 (4 weeks post-induction) |
| 4 | 10 |
| 5 | 20 |
| 6 | 35 |
| 7 | 40 |
| 8 | 45 |

Immunochemical Examination of Reduction of Expression of iNOS and COX-2 in Treated Mice:

MM cells are known to express inducible nitric oxide synthase (iNOS) which catalyzes the production of the inflammatory mediator nitric oxide (NO). NO has been shown to protect MM cells from apoptosis by nitrosylating and inactivating caspase 9, an essential protein in the apoptotic pathway (See, Ellerhorst J. A. et al., Oncol. Rep., 2010; 23(4):901-907; Salvucci O. et al., Cancer Res., 2001; 61:318-326 and Torok N. J. et al., Cancer Res., 2002; 62:1648-1653). Likewise, MM cells are known to overexpress COX-2, which synthesizes prostaglandin E2 (PGE2). PGE2 stimulates overexpression of Bc1-2 protein. Bc1-2 protein acts to bind and inhibit the pro-apoptotic proteins BCL-2 associated x protein (Bax) and BCL-2 antagonist killer 1 protein (BAK) which blocks apoptosis (See, Chipuk J. E. and Green D. R., Trends in Cell Biology, 2008; 18(4):157-164 and Fosslien E., Ann. Clin. Lab. Sci., 2000; 30(1):3-21). Because both iNOS and COX-2 are known to inhibit apoptosis in MM cells, these proteins are used as reliable biomarkers for the progression and invasiveness of melanoma cells.

Histological samples of the melanoma in an experimental mouse were taken during the course of treatment with the pharmaceutical composition of Example 3.

Formalin-fixed paraffin-embedded sections of mouse foot pad melanoma tissue were examined for iNOS and COX-2 expression by immunohistochemistry (IHC) using an anti-iNOS rabbit monoclonal antibody (1:50) (Labvision, CA, USA) and anti-COX-2 mouse monoclonal antibody (1:50) (Transduction Laboratories, Lexington, Ky.).

Tissue sections were de-paraffinized and rehydrated, then placed in a 0.01 M citrate buffer, pH 6, and microwaved intermittently for a total of 20 min. After cooling, the slides were placed in 3% aqueous H₂O₂ for 30 min. An avidin-biotin-peroxidase complex (ABC) kit (Vectastain, Vector Laboratories) was then used for antigen detection. After 30 min of blocking in 1% BSA, the primary antibody was applied overnight at 8° C., followed by a 30 min incubation with secondary biotinylated antibody, and the ABC reagent.

The immunolabeling was developed with the chromogen 3-amino-9-ethylcarbazole for 6 min. Hematoxylin was applied as a counter stain. A colon carcinoma with known COX2 and PPARG expression was chosen as a positive control. Normal tissue samples of the foot pad of control animals were considered as negative controls. Immunolabeling was scored separately for two variables: (1) number of iNOS and COX-2 positive cells; and (2) overall intensity of immunoreactivity of the positive cells. Briefly, scoring for number of positive cells was defined as follows: “0”, <5% positive cells; “1”, 5-25% positive cells; “2”, 25-75% positive cells; “3”, greater than 75% positive cells. Intensity scoring was defined as follows: “0”, no staining; “1”, weak staining; “2”, moderate staining; and “3”, intense staining.

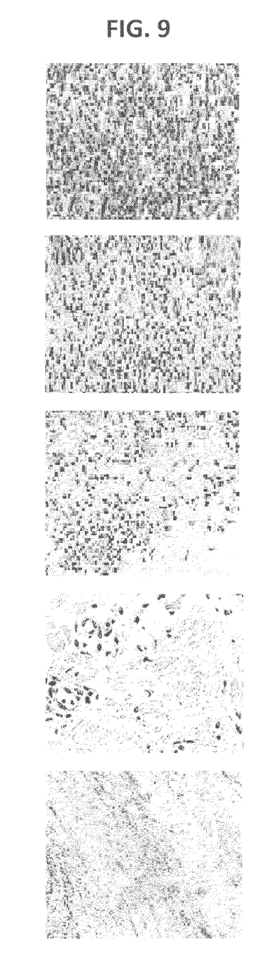

The results of immunohistochemistry are also depicted in FIG. 9 (COX-2 staining) and (iNOS staining) as shown in Table 2 as follows:

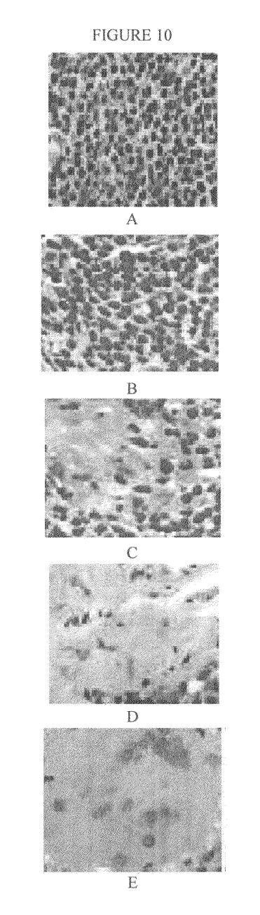

TABLE 2

| FIGS. 9 and 10 | Detailed Description |
|---|---|
| A | No treatment |
| B | 7 days treatment |
| C | 14 days treatment |
| D | 21 days treatment |
| E | Control (healthy animal) |

The decrease in the area density of stained areas in FIGS. 9 and 10 showed the marked reduction in the expression of iNOS and COX-2.

### Example 5

### Inhibition of Melanoma Progression in a Mouse Foot Pad Model

In this study, mice were used to test whether the pharmaceutical composition of Example 3 could inhibit the progression of melanoma.

An individual experimental mouse was obtained, maintained, and inoculated with B16 cells as described in Example 4. An early-stage melanoma was visible seven days after inoculation with B16. Beginning on day 8, injections of 100 μl of the pharmaceutical composition of Example 3 were administered daily.

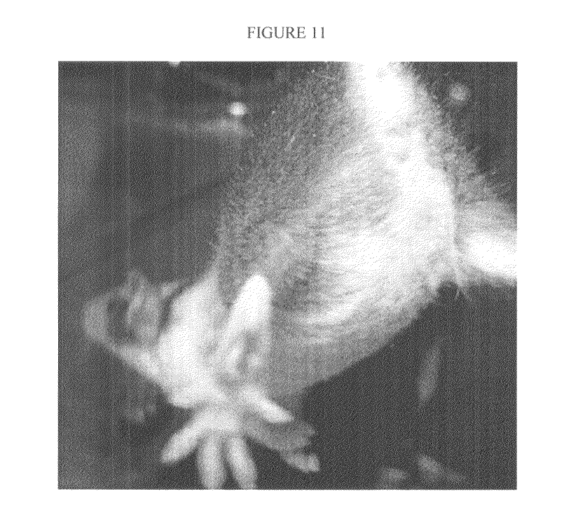

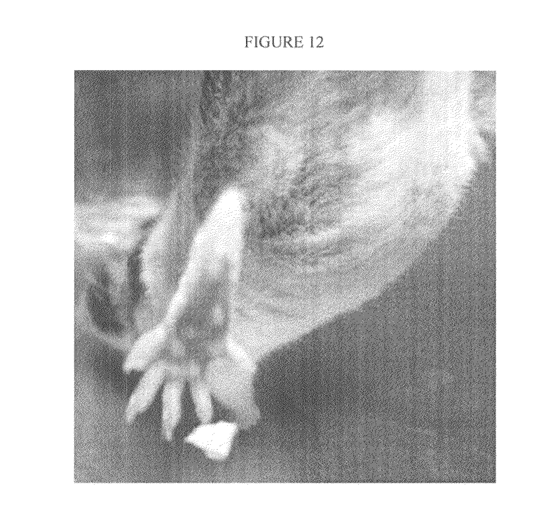

Development of the early stage melanoma can be observed in FIG. 11. FIG. 12 shows the foot pad of the same mouse after 20 days treatment. Comparison of FIG. 12 to FIG. 2 (21 days post-innoculation, no treatment) demonstrated the effectiveness of the pharmaceutical composition of Example 3 in inhibiting progression of the melanoma.

### Example 6

### Treatment of Traumatic Brain Injury (TBI)

Traumatic brain injury (TBI) is caused by a head injury such as a blow to the head, concussive forces, acceleration-deceleration forces, or a projectile that can result in lasting damage to the brain and affects up to 10 million patients worldwide each year. It may occur both when the skull fractures and the brain is directly penetrated (open head injury) and also when the skull remains intact but the brain still sustains damage (closed head injury).

TBI is graded as mild (meaning a brief change in mental status or consciousness), moderate, or severe (meaning an extended period of unconsciousness or amnesia after the injury) on the basis of the level of consciousness or Glasgow coma scale (GCS) score after resuscitation. The GCS scores eye opening (spontaneous=4, to speech=3, to pain=3, none=1), motor response (obeys=6, localizes=5, withdraws=4, abnormal flexion=3, extensor response=2, none=1), and verbal response (oriented=5, confused=4, inappropriate=3, incomprehensible=2, none=1). Mild TBI (GCS 13-15) is in most cases a concussion and there is full neurological recovery, although many of these patients have short-term memory and concentration difficulties. In moderate TBI (GCS 9-13) the patient is lethargic or stuporous, and in severe injury (GCS 3-8) the patient is comatose, unable to open his or her eyes or follow commands. Patients with severe TBI (comatose) have a significant risk of hypotension, hypoxaemia, and brain swelling. If these sequelae are not prevented or treated properly, they can exacerbate brain damage and increase the risk of death.

Symptoms of TBI may include, but are not limited to, memory or concentration problems, dizziness or loss of coordination, slurred speech, sensory problems (e.g., blurred vision, ringing in the ears, etc.), headache, mood changes or mood swings, depression, anxiousness, and the like (See, Traumatic Brain Injury: Hope Through Research, 2002, the National Institute of Neurological Disorders and Stroke (NINDS)).

TBI is characterized by two injury phases, primary and secondary. The primary brain injury is the direct injury to the brain cells incurred at the time of the initial impact. This results in a series of, biochemical processes leading to secondary brain injury (See, e.g., Veenith T. et al., World Journal of Emergency Surgery, 2009; 4:7-12). The secondary brain injury is caused by a dynamic interplay between ischemic, inflammatory and cytotoxic processes. One of the most significant factors causing secondary brain injury is the excessive release of excitotoxins such as glutamate and aspartate that occurs at the time of the primary brain injury (See, Veenith T. et al., World Journal of Emergency Surgery, 2009; 4:7-12), which act on the N-methyl-D-aspartate channel, altering cell wall permeability with an increase in intracellular calcium and sodium and activation of calcineurin and calmodulin. This ultimately, leads to destruction of the axon (See, Veenith T. et al., World Journal of Emergency Surgery, 2009; 4:7-12 and Smith D. H. et al., The Neuroscientist, 2000; 6:483-495). Potassium is also released from the cells, and, iIn an attempt to restrict the ionic imbalance, absorbed by astrocytes causing swelling of these cells and ultimately cell death (See, Veenith T. et al., World Journal of Emergency Surgery, 2009; 4:7-12).

Apoptosis is recognized as an important factor in secondary brain injury (See, e.g., Rink A. et al., Am. J. Pathol., 1995; 47(6):1575-1583 and Veenith T. et al., World Journal of Emergency Surgery, 2009; 4:7-12). Cells undergoing apoptosis die without membrane rupture and therefore elicit less inflammatory reactions. This is in contrast to the cells undergoing necrosis (See, Tolias C. M. et al., NeuroRx, 2004; 1(1):71-9 and Veenith T. et al., World Journal of Emergency Surgery, 2009; 4:7-12). This suggests that neuronal apoptosis after TBI may be a protective response by the brain in order to remove injured cells without affecting the remaining brain tissue (Raghupathi R., Brain Pathol., 2004; 14:215-222 and Veenith T. et al., World Journal of Emergency Surgery, 2009; 4:7-12)

The apolipoprotein epsilon (APOE) gene is important in the neuronal response of the brain to injury and in the subsequent repair processes. There are three different variants (ε2, ε3, and ε4) to this gene. The variant ε4 is associated with a poor outcome in cognitive dysfunction and functionality following brain injury rehabilitation (Crawford F. C. et al., Neurology, 2002; 58(7):1115-1118 and Veenith T. et al., World Journal of Emergency Surgery, 2009; 4:7-12). It is also associated with a rapid cognitive decline in Alzheimer's disease (Wilson M. et al., Br. J. Anaesth., 2007; 99(1):43-48 and Veenith T. et al., World Journal of Emergency Surgery, 2009; 4:7-12).

In this study, mice were used to test whether the pharmaceutical composition of Example 3 could treat the symptoms associated with traumatic brain injury (TBI).

Morris Water Maze Test:

The Morris water maze test (http://en.wikipedia.org/wiki/Morris water navigation task) was used to investigate the effect of treatment with the pharmaceutical composition of Example 3 on the rate at which animals recovered spatial memory after suffering TBI.

Briefly, a video camera was placed above the center of a 180 cm diameter circular pool filled with water to capture images of the swimming animal for tracking purposes to determine the time and efficiency with which the animals could find a learned escape platform hidden 1.5 cm below the surface of the water, the location of which can normally be identified by a mouse only by reliance on spatial memory.

Mice: Ten mice, obtained and maintained as in Example 4, were divided into experimental and control groups, 5 mice per group. Prior to induction of TBI, all mice were trained to find the escape platform of the Morris water maze test so that the rate of post-TBI memory recovery could be determined.

Induction of TBI: The mice in the experimental group were briefly anesthetized with either in an exicator and then placed on the WDM platform. Animals were immobilized using magnetic clips.

A 200 g weight was drop-released from a height of 4 cm; inducing a focal blunt injury over an intact skull of the mouse. The impact induced a closed head injury with profound neuroinflammatory response within the intrathecal compartment, including bleeding and brain swelling.

Treatment of TBI: The mice in the experimental group received daily intraperitoneal injections of 100 μA of the pharmaceutical composition of Example 3 and were evaluated for memory restoration using the Morris water maze test. Mice suffering from TBI and receiving daily administration of 100 μl of the pharmaceutical composition of Example 3 for 5 to 45 days showed significant improvements in spacial memory, evidenced by the time required to find the escape platform in the Morris water maze test.

Healthy mice in the control group (no TBI, no treatment) found the escape platform (i.e. remembered surroundings), on average, 2.2 times faster than did non-treated animals inflicted with TBI. Treated animals inflicted with TBI found the escape platform 3.8 times faster than did non-treated animals inflicted with TBI; faster than the animals in the control (uninjured) group.

The efficacy of the pharmaceutical composition of Example 3 in treating the signs and symptoms of TBI was further documented by visual inspection of the exposed brains of healthy (non-injured), injured and non-treated, and injured and treated mice. A photographic record of the visual inspection of the subject animals is presented in FIG. 12 as shown in Table 3 as follows:

TABLE 3

| Figure | Detailed Description |
|---|---|
| 13A | Exposed brain of test animal about 5 min. post-trauma |
| 13B | Exposed brain of healthy (non-traumatized) test animal |
| 13C | Exposed brain of a traumatized test animal 14 days post-trauma |
| 13D | Exposed brain of traumatized test animal 14 days after treatment |
| | started within one day of injury (inured animal treated for 14 |
| | days, then skull opened) |

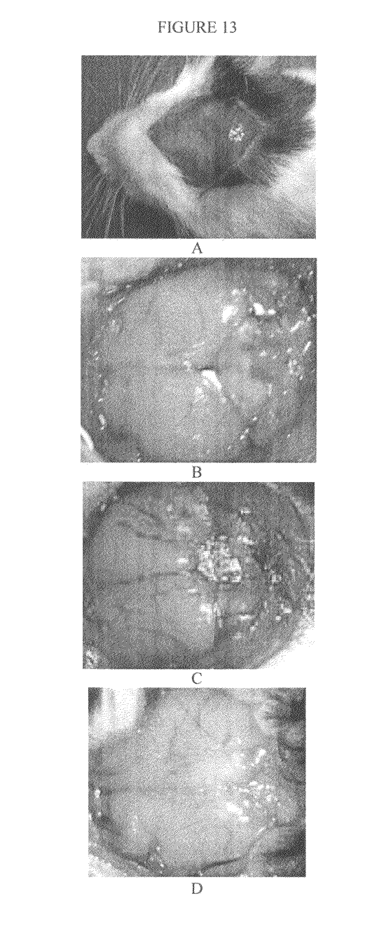

The brain of FIG. 13 D was judged to be indistinguishable from the healthy brain of FIG. 13 B.

### Example 7

### Treatment of Post-Traumatic Alopecia Induced by Cranial Injury

Traumatic alopecia (i.e., hair loss) can be caused by many different types of physical and chemical injury to the hair and scalp. These injuries often result in the increased destruction, the defective regeneration, or the defective formation of hair follicles.

In this study, mice were used to test whether the pharmaceutical composition of Example 3 could treat post-traumatic brain injury alopecia.

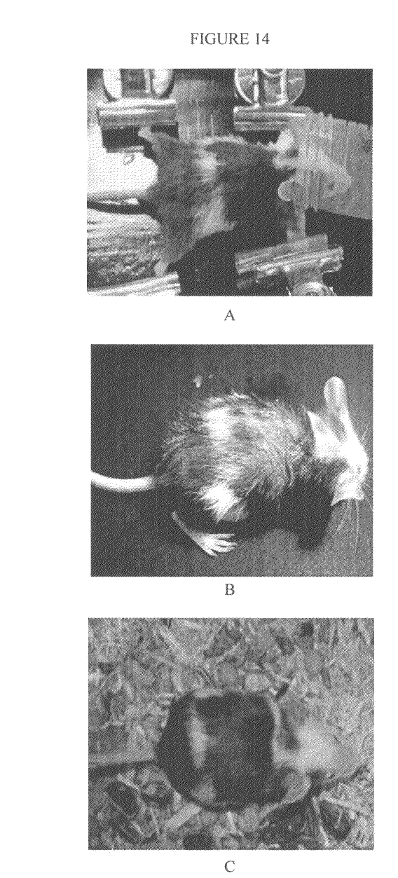

In awareness of the possible occurrence of post-traumatic alopecia, the mice in the experimental group of Example 6 were examined for post-TBI (post-trauma) hair loss. FIG. 14 documents the development and resolution of post-TBI alopecia in an experimental animal as shown in Table 4 as follows:

TABLE 4

| Figure | Detailed Description |
|---|---|
| 14A | Experimental animal before infliction of TBI |
| 14B | Same experimental animal 10 days after infliction of TBI |
| 14C | Same experimental animal after 7 days treatment that began |
| | 14 days post-TBI and continued for 21 days. Post-traumatic |
| | alopecia was reduced. |

### Example 8

### Treatment of Skin Wrinkling

Facial muscles (also known as musculi facials, or mimetic muscles), are a group of striated muscles innervated by cranial nerve VII, also known as the facial nerve. They are subcutaneous (meaning just under the skin) muscles that control facial expression. They generally originate on bone, and insert on the skin of the face. A “facial expression”, which is a form of nonverbal communication, results from one or more motions or positions of the muscles of the face. The muscles that allow this complex communication are located in superficial positions along the face, including muscles around the eyes, mouth, nose and forehead, the scalp and the neck (Table I.) The largest group of facial muscles is associated with the mouth. Smaller groups of muscles control movements of the eyebrows and eyelids, the scalp, the nose, and the external ear. During a spontaneous smile, for example, the corners of the mouth lift up through movement of the zygomaticus major muscle, and the eyes crinkle, causing “crows feet” through contraction of the orbicularis oculi muscle.

TABLE 5
Muscles of Facial Expression

| Muscle | Origin | Insertion | Action |
|---|---|---|---|
| Frontalis | Galea | Skin of eyebrows | Raises eyebrows, |
| | aponeurotica | and nose | wrinkles forehead skin |
| Orbicularis | Frontal and | Skin of eyelid | Blinking, squinting, |
| oculi | maxillary bone | | forceful closing of |
| | | | eyelids |
| Orbicularis | Fibers of other | Muscles and skin | Closes and protrudes |
| oris | mouth muscles | at angle of the | lips |
| | | mouth | |
| Platysma | Pectoralis and | Lower border of | Depresses mandible, |
| | deltoid fascia | the mandible, | draws angle of mouth |
| | | mouth skin and | downward, tightens |
| | | muscle | skin of the neck |

A “wrinkle” is a ridge or crease of the skin surface caused by the effects of facial muscles. Wrinkling in skin, including, but not limited to, crows feet around the eye, undereye wrinkles, neck wrinkles, “smile lines”, “parentheses lines”, and wrinkles around the lips, is caused by a number of factors, including habitual facial expressions, aging, sun damage, smoking, and poor hydration. Wrinkles can be present as either fine surface lines or deep furrows.

Some subjects will do just about anything to reduce or eliminate the appearance of wrinkles Consequently, a number of products and procedures have been developed to rejuvenate the appearance of skin. Many of these products and procedures have undesirable side effects.

In this study, mice were used to test whether the pharmaceutical composition of Example 3 could reduce skin wrinkling.

Squalene Monohydroperoxide:

Topical application of squalene monohydroxide was used to induce skin wrinkling Squalene-monohydroperoxide (Sq-OOH) was prepared from squalene (sigma cat# S3626) using the experimental protocol described by Chiba, K. et al. (Experimental Dermatology, 1999; 8:471-479), slightly modified for our needs. Briefly, 10 mL of squalene in a 50 mL beaker was irradiated for 2 h in a biosafety cabinet using a Dennalight 80 UV Phototherapy UVB 311 phototherapy system (Munich, Germany), providing UV radiation of 340-440 nm, positioned at a distance of 30 cm. UVB irradiation was carried out using a PLS9w/01 (DRH060) UVB light source (Philips, Aachen, Germany).

Protocol:

Ten mice obtained and maintained as in Example 4 were divided evenly into two groups, experimental (5 mice) and control (5 mice). Approximately 4 cm² of right lateral skin of all mice was depilated using Hibros depil sport depilatory cream and sterile MasterAmp™ Buccal Swab Brush (Epicentre Biotechnologies cat# MB1OOSP). To induce skin wrinkling in the experimental group, MasterAmp™ brushes soaked in SqOOH were used for daily topical application of squalene-monohydroperoxide to exposed skin for up to 3 weeks. In the control group, 200 μl were applied daily to the exposed skin. On day 7, changes in the skin were photographed using Nikon Coolpix 14.0 megapixel digital camera. Mice in the experimental group received daily intraperitoneal injections of 100 μl of the pharmaceutical composition of Example 3 for up to 45 days.

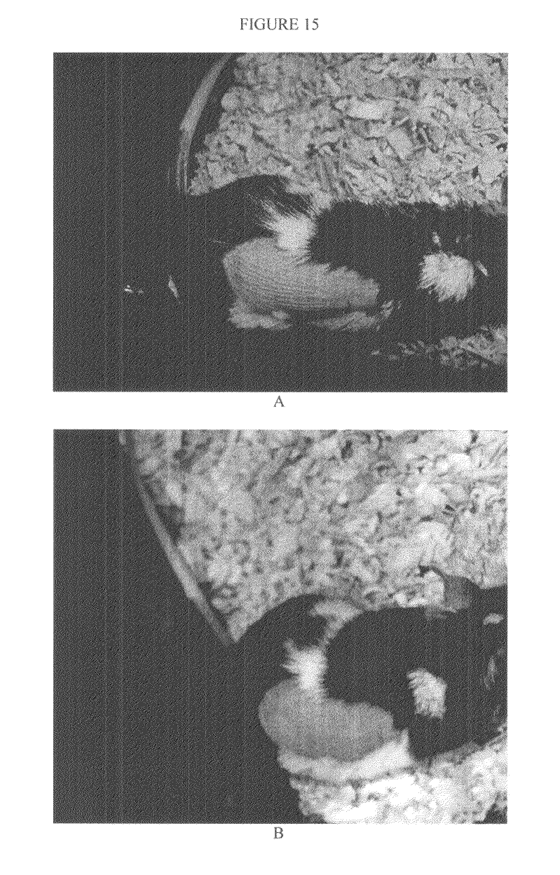

FIG. 15A shows a mouse from the experimental group with pronounced skin wrinkling FIG. 15B shows the same mouse after 7 days treatment with the pharmaceutical composition of Example 3, in which skin wrinkling was reduced.

### Example 9

### Mouse Gerontology Study

Aging is considered to be a multifactorial process influenced by both genetic and environmental components. Although a number of different theories of aging have been proposed, none explains the aging process in its entirety (See, Mercado-Saenz S. et al., Brazilian Archives of Biology and Technology, 2010; 53(6):1319-1332). Despite the number of theories, it is generally accepted that aging primarily is associated with two processes, progressive cell degeneration and the loss of cell regenerative capacity. Progressive cell degeneration is principally related to the incomplete suppression of the production and elimination of reactive oxygen species (ROS) and to the glycosylation of proteins (See, Mercado-Saenz S. et al., Brazilian Archives of Biology and Technology, 2010; 53(6):1319-1332). Loss of cell regenerative capacity is determined genetically, for example, by the shortening of telomeres due to the suppression of telomerase, the activation of a mechanism related to age that stimulates heat shock proteins, the accumulation of mutations in the genome of somatic cells which leads to the development of neoplasias and the decrease of organ functions, and by processes of apoptosis (See, Bushell W. C., Ann. NY Acad. Sci., 2005; 1057:28-49; Knaposwski J. et al., J. Physiol. Pharmacol., 2002; 53:135-146; Weng N. P. et al., Immunol. Rev., 1997; 160:43-54 and Mercado-Saenz S. et al., Brazilian Archives of Biology and Technology, 2010; 53(6):1319-1332).

### Experimental

In this study, mice were used to test whether the pharmaceutical composition of Example 3 could affect overall life expectancy.

Protocol:

Twenty mice obtained and maintained as in Example 4 were divided into two groups, experimental and control, consisting of ten mice each. The mice were segregated by gender. Mice in each group received the same diet, described in Example 4, and were housed under the same conditions. Mice in the experimental group were administered daily intraperitoneal injections of 100 μl of the pharmaceutical composition of Example 3. Mice in the control group were administered daily injections of 100 μl of HEPES and penicillin/streptomycin-free OWS.

Results:

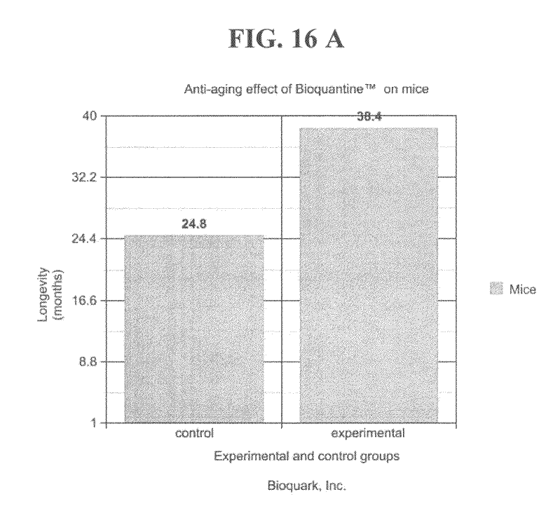

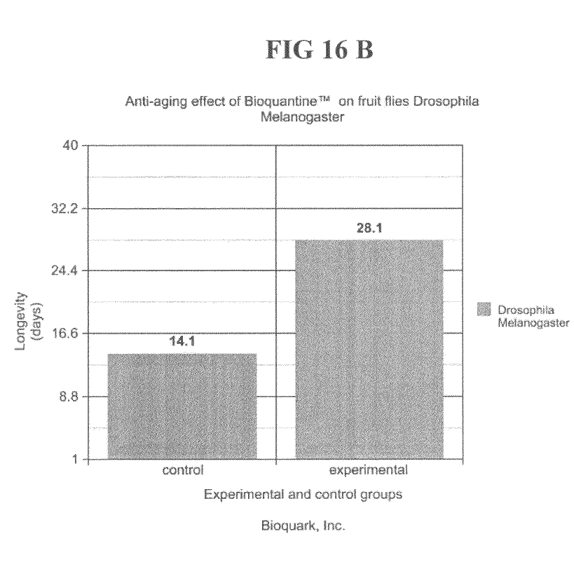

The results of this experiment are presented in FIG. 16A. Mice that received daily injections of the pharmaceutical composition of Example 3 survived on average, about 50% longer than the mice in the control group. Notably, no local inflammatory response (e.g. abscesses) and no behavioral responses were observed in the mice that received daily injections of the pharmaceutical composition of Example 3.

### Example 10

### Invertebrate Gerontology Study

In this study, Drosophila melanogaster was used to test whether the pharmaceutical composition of Example 3 could affect overall life expectancy. Drosophila melanogaster is a well-established model system for human aging. The conservation of human genes in Drosophila melanogaster allows the functional analysis of orthologues implicated in human aging and age-related diseases (See, Brandt A. and Vilcinskas A., The Fruit Fly Drosophila melanogaster as a Model for Aging Research, Advances in Biochemical Engineering/Biotechnology, DOI:10.1007/10_{—}2013_{—}193, Springer-Verlag Berlin Heidelberg 2013). For example, Drosophila melanogaster models have been developed for a variety of age-related processes and disorders, including stem cell decline, Alzheimer's disease, and cardiovascular deterioration (See, Brandt A. and Vilcinskas A., The Fruit Fly Drosophila melanogaster as a Model for Aging Research, Advances in Biochemical Engineering/Biotechnology, DOI:10.1007/10_{—}2013_{—}193, Springer-Verlag Berlin Heidelberg 2013).

Drosophila melanogaster:

Drosophila BioKit was purchased from Carolina Biological Company (CBC cat#17-1960). Drosophila were cultured in glass culture vessels supplemented with formula 4-24 Instant Drosophila Medium (CBC cat#17-3200). Drosophila were anesthetized in an empty vial (CBC cat#17-3120) using carbon dioxide tablets (CBC cat#17-3037). The anesthetized flies were placed in a row on a white note card and examined with a microscope at a magnification 15×. The sex of Drosophila was distinguished by examination of the genital organs using an optical microscope at a magnification 15×. Male genitalia were surrounded by heavy, dark bristles, which do not occur on the females. Using a sorting brush (CDC cat#17-3094) male Drosophila were separated from females. Male and female Drosophila were each separated into two gender-specific groups, experimental and control, comprised of 100 Drosophila each.

Protocol:

Drosophila in each of the experimental groups were sustained on feed comprised of 10 g of 4-24 (Drosophila Medium, Carolina Biological Company, cat#17-3200) diluted in 10 mL of the pharmaceutical composition of Example 3. Drosophila in the control groups were sustained on Formula 4-24.

Results:

The results of this experiment are presented in FIG. 16B. The life span of Drosophila sustained on feed supplemented with the pharmaceutical composition of Example 3 was twice that of Drosophila sustained on feed that was not supplemented with the pharmaceutical composition of Example 3.

### Example 11

### Reprogramming of Normal and Cancerous Human Cells to iPSC-Like Cells

In this study, normal human cells were electroporated with Xenopus laevis oocytes in the final stage of maturity in order to test whether the pharmaceutical composition can influence regulatory mechanisms involved in reprogramming differentiated human cells into iPSC-like cells.

Cell Lines:

Human bone marrow stromal Cells (BMSCs) and stably transfected GFP-expressing BMSCs (BMSC_{GFP}) were provided by Tulane University Center of Gene Therapy. Prior to release from the source, two trials of frozen, passage-1 cells were analyzed over three passages for colony forming units, cell growth, and differentiation into fat, bone, and chondrocytes. The BMSC and BMSC_{GFP} were cultured in Dulbecco's modified Eagle's Medium (DMEM; Sigma), supplemented with 10% fetal bovine serum (FBS; Gibco) and 1% penicillin/streptomycin (Gibco) and cultured in 25 cm² (T25) flasks at 37° C. with 5% C0₂. At day 4, the cultures were washed with phosphate buffered saline (PBS; Sigma) to remove the non-adherent cells and further expanded until about 80% confluence, when they were harvested and expanded in 75 cm² flasks.

Human normal foreskin fibroblasts (BJ cells) from American Type Culture Collection (ATCC) were maintained at 37° C. and 5% C0₂ in T25 culture flasks in 5 ml of Eagle's Minimum Essential Medium (EMEM; ATCC) supplemented with 10% PBS, 1 mM sodium pyruvate, 0.1 mM nonessential amino acids (NEAA), and 1% penicillin/streptomycin.

Human subcutaneous pre-adipocytes (HPA) from ScienCe II Research laboratories were cultured at 37° C. and 5% C02 in T25 flasks coated with 0.01% poly-lysine (Sigma) and containing 5 ml of specially formulated pre-adipocyte medium (PAM; ScienCells); PAM was supplemented with 5% FBS, 1 mM sodium pyruvate, 0.1 mM NEAA, and 1% penicillin/streptomycin.

Human peripheral blood CD4+ T-lymphocytes (CD4TLs) from Lonza Group, ltd. (pathogen-free poietics® CD4TLs) were maintained as a cell suspension in T25 culture flasks at 37° C. and 5% C0₂ in 5 ml of lymphocyte growth medium-3 (LGM-3®, lonza Group ltd.) supplemented with 10% FBS, 1 mM sodium pyruvate, 0.1 mM non-essential amino acids, 1% penicillin/streptomycin, and 50 ng/ml recombinant human Interleukin-4 (R&D Systems).

Human buccal mucosa cells were obtained from healthy human subjects approximately 1 hour before the co-electroporation procedure. Subjects abstained from drinking coffee for 1 hour before collection. Subjects' mouths were rinsed twice with listerine® followed by sterile distilled water before swabbing. Cells were collected by swabbing firmly on the inside of the cheek 20 times on both sides using a MasterAmp™ Buccal Swab Brush (Epicentre Biotechnologies). The brush holding cheek cells was placed into a 50 ml centrifuge tube filled with 20 ml of sterile filtered PBS (Sigma) containing 1% penicillin/streptomycin. The sample was vigorously twirled for 30 sec and then centrifuged at 200×g for 7 min. Pelleted cells were resuspended in 5 ml of serum-free DMEM (ATCC) supplemented with 1 mM sodium pyruvate, 0.1 mM NEAA, and 1% penicillin/streptomycin. Buccal mucosa cells were kept in a refrigerator at 4° C. before use.

Human cervical carcinoma (Hela) cells (routinely maintained at the Bioquark, Inc. facility) were grown at 37° C. and 5% C0₂ in T25 flasks filled with 5 ml of Eagle's essential medium (ATCC) supplemented with 10% FBS, 1 mM sodium pyruvate, 0.1 mM NEAA, and 1% penicillin/streptomycin.

Human breast adenocarcinoma (MCF-7) cells from ATCC were maintained in Eagle's Minimum Essential Medium supplemented with 10% FBS, 1 mM sodium pyruvate, 0.1 mM NEAA, 1% penicillin/streptomycin, and 0.01 mg/ml recombinant human insulin (Eli Lilly; a gift from North-Suburban Pharmacy, Skokie, Ill.). Irradiated mouse embryonic fibroblasts (iMEF; American R&D Systems) were grown at 37° C. and 5% C0₂ in non-pyrogenic, sterile 25 cm², 0.2 μm ventilated cell culture flasks (T25; Corning) containing 5 ml of high glucose DMEM (Millipore) supplemented with 10% FBS, 1 mM sodium pyruvate, 0.1 mM NEAA, and 1% penicillin/streptomycin.

Co-electroporation of Xenopus laevis Oocytes with Human Cells:

Forty to fifty fresh oocytes from suspensions obtained in Example 1 with ≧90% viability (oocytes showing abnormal pigment distribution or signs of damage of equatorial band, patchy gray membranes during the defolliculation process were discarded) were placed in sterile Gene Pulser electroporation cuvettes (Bio-Rad) prefilled with 400 μl of serum-free DMEM containing 1.0×10⁵-1.5×10⁵ cells/ml of human cells in suspension. Cuvettes were filled to 800 μl with serum-free DMEM and then placed into the shocking chamber. Co-electroporation of frog oocytes with the suspension of human cells was conducted using the following parameters: 150 v/cm/25 μF/7 pulses, with time constant at 0.5-0.7 msec. After electroporation, cuvettes containing oocytes and the human cells were incubated at 17° C. for three hours to recover. The human cells were transferred to T25 culture flasks containing iMEF feeder cells for culturing.

Culturing of Human Cells Following Co-Electroporation:

The co-electroporated human cells were cultured at 37° C. on iMEF feeder cells in 0.1% gelatin-coated (gelatin from Sigma) T25 culture flasks containing 5 ml of specially formulated Embryomax® DMEM culture medium (Millipore). Medium was supplemented with 15% FBS, 1 mM sodium pyruvate, 0.1 mM NEAA, 1% penicillin/streptomycin, 100 μM beta-mercaptoethanol (Gibco), and 1000 U/ml ESGRQ® (Millipore). To maintain the cells in an embryonic stem cell-like state, 1000 U ESGRQ® per 1.0 ml of tissue culture media was required. After formation of clusters, the human cells were separated from the feeder cells using the differential sedimentation technique previously described by Doetschman (Doetschman T., Gene Targeting in Embryonic Stem Cells: A Laboratory Handbook, San Diego, Calif., Academic Press, 2002), which removed>99% of contaminating feeder cells from the electroporated human cell suspension. Trypsinized (trypsin from Sigma) human cell cultures containing iMEFs were centrifuged at 200×g, resuspended in 10 ml of complete ES culture medium, and transferred to a new T25 cell culture flask for 30 minutes at 37° C. Following incubation, the culture medium containing mostly human cells was transferred to a new T25 culture flask for 1 hour at 37° C. to remove all remaining fibroblast feeders. Following the second incubation, the culture medium containing the human cells was removed, and the cells were counted, centrifuged again at 200×g, and resuspended in the ES culture medium.

Subculturing:

After separation from the feeder cells, the human cells were plated on T25 culture flasks containing either iMEF feeder cells or feeder-free StemAdhere™ pluripotency substrate (Primorigen Biosciences). Subcultured human cells were grown in NutriStem™ (Stem Gent).

Calculation of Reprogramming Efficacy:

Fluorescent immunohistochemically detectable expression of the Nanog gene by cells derived from CD4T1s occurred between 12 h-24 h following co-electroporation with Xenopus laevis oocytes. This expression preceded the formation of tight iPSC-like clusters, making it possible to determine the efficiency of reprogramming by calculating the proportion of cells expressing Nanog gene. The mean for the reprogramming efficiency was calculated by counting the total number of Nanog-positive cells per specimen in each T25 flask (3-4 times), subtracting the number of nonspecific binding sites in the control flasks, dividing by the original number of cells having undergone co-electroporation and multiplying by 100%. The standard deviation of the mean was also calculated.

Cryopreservation of Reprogrammed Cells:

Cells were cryopreserved using a standard slow-cooling freezing method (Peterson S. et al., Human Stem Cell Manual, A Laboratory Guide, Academic Press, 2007). One ml of cells was gently resuspended in 1.5 ml cryovials (Nalgene) containing 0.5 mL of 2×hES cell freezing medium (60% FBS, 20% hES cell culture medium, and 20% dimethyl sulfoxide). Cryovials were transferred to 5100 Cryo 1° C. Freezing Container (Nalgene), refrigerated at −80° C. overnight and then rapidly transferred to liquid nitrogen refrigeration units.

Trans-differentiation into Neuronal Progenitor Cells:

After formation of clusters, reprogrammed cells derived from Human subcutaneous pre-adipocytes (HPA) were separated from the feeder layer using the Doetschman differential sedimentation technique and were dissociated enzymatically using collagenase IV (Sigma; 200 U/mL) for 30 min at 37° C. generating a cell suspension containing small cell aggregates and single cells. Cell culture conditions for growing neural progenitor cells (NPs) from embryonic stem cells were employed (Axell M. Z. et al., J. Neurosci. Methods, 2009; 184:275-284). The cells were washed in warm Neurobasal A medium (GibcoBRL/Invitrogen), pelleted and resuspended in pre-warmed (37° C.) standard human embryonic stem cell culturing medium (hESC) supplemented with following growth factors and neuronal and other supplements: fibroblast growth factor-2 (10 ng/mL), epidermal growth factor (20 ng/mL), 1% B27, 1% N2, 1% penicillin/streptomycin, 1% 1-glutamine, 1% non-essential amino acids (NEAA), 0.2% beta-mercaptoethanol, and 20% Knockout Serum Replacement (all media components from Gibco-BRL/Invitrogen). The HPA-derived cells in suspension were then seeded at high cell density (150-200×10³ cells/cm²) onto BD BioCoat™ and laminin-coated 150 mm petri dishes (Becton Dickinson), and the medium was supplemented with hESC medium Embryomax® DMEM culture medium (Millipore cat.#SLM-220-M, Danvers, Mass., USA) and 4 ng/ml fibroblast growth factor-2. Proliferating HPA-derived neural progenitors were observed in 8-10 days. The neural rosettes were dissociated by short (5-10 min) collagenase IV treatment into single cells and re-seeded under the same conditions, thus generating a monolayer population of proliferating neural progenitors.

Qualitative Assessment of Colony Morphology:

Assessment of colony morphology (resemblance to iPSe colonies) was performed by Dr. Nikolai Strelchenko, PhD of the hESC Research Lab at Reproductive Genetics Institute, Chicago, Ill., USA and Dr. Arshak Alexanian, V M D, PhD, of the Department of Neurosurgery, Neuroscience Research Laboratories, Zablocki Veterans Affairs Medical Center and of Medical College of Wisconsin, Milwaukee, Wis., USA.

Alkaline Phosphatase (AP) Staining and Fluorescent Immunocytochemistry:

AP is a phenotypic marker of pluripotent stem cells (PSCs), including undifferentiated embryonic stem cells (ESCs), induced pluripotent stem cells (iPSCs), and embryonic germ cells (EGCs). While AP is expressed in most cell types, its expression is highly elevated in PSCs. AP staining has therefore been used to differentially stain PSCs to easily distinguish them from mouse embryonic fibroblasts (MEFs) used as feeders and parental fibroblasts commonly used in reprogramming experiments.

Histochemical staining for alkaline phosphatase (AP) was conducted using the Vector® Blue Alkaline Phosphatase Substrate Kit III (Vector Laboratories, Inc.). Expression of several pluripotency factors was assayed using fluorescent immunohistochemistry conducted at room temperature. Samples from all populations of human cells in T25 culture flasks went through the following steps: (a) the growth medium was removed, (b) washed three times with PBS, (c) fixed in −10° C. methanol, (d) washed three times with PBS, (e) incubated for 20 min in 10% normal serum, (f) incubated for 60 min. in primary antibody diluted in 1.5% normal serum, (g) washed three times with PBS, (h) incubated for 45 min. in the dark with secondary antibody diluted in 1.5% normal serum, (i) washed three times with PBS and left in 3rd rinse, (j) examined under an inverted-phase contrast fluorescent microscope, (k) PBS replaced with the anti-fading reagent 2% DABCO (Sigma), and (1) processed T25 flasks with specimens were sealed with parafilm, wrapped in aluminum foil and stored at 4° C.

The primary and secondary antibodies and normal sera (2.5 μg/mL) included polyclonal goat anti-Oct3/4 IgG, polyclonal goat anti-Nanog IgG, polyclonal goat anti-Sox-2 IgG, monoclonal mouse anti-TRA-1-60 IgG, monoclonal mouse anti-SSEA-1 IgM, polyclonal goat anti-Rex-1 IgG, goat-anti mouse IgM-TR, donkey-anti-mouse IgG-FITC, donkey anti-goat IgG-FITC, donkey antigoat IgG-TR, normal donkey serum, and normal goat serum (all from Santa Cruz Biotechnology, Inc). Anti-sera to the following were used to analyze formation of neural progenitor cells:nestin (1:500 dilution, BD Pharmingen), beta-3 tubulin monoclonal antibody (B3T; 10 μg/ml; Pierce antibodies), neural cell adhesion molecule (NCAM), 1:500 dilution (Abcam), glial fibrillary acidic protein (GFAP, 1:250 dilution (Abcam). DNA staining was performed using 4′,6-diamidino-2-phenylindole, 4′,6-diamidinophenyl-indole (DAPI; Santa Cruz Biotechnology, Inc.).

Control Experiments:

The control experiments described in the following Table 6 were used to test for the effect of the presence of human cells, oocytes, feeder cells, co-electroporation, and the electroporate on reprogramming (expression of Nanog detected using fluorescent immunohistochemistry).

TABLE 6

| | | | | | Post-Electroporation | | |
|---|---|---|---|---|---|---|---|
| | Electroporation Conditions | | | | Incubation Conditions | | |
| | Human | | iMEF | | Human | | Nanog |
| Control | Cells* | Oocytes | Cells* | Electroporation | Cells* | Other | Expression |
| a | ✓ | | | ✓ | ✓ | | Negative |
| b | ✓ | ✓ | | | ✓ | | Negative |
| c | ✓ | | | | | | Negative |
| d | | ✓ | | ✓ | ✓ | Electroporate | 0.4%^{§} |
| | | | | | | from | |
| | | | | | | oocytes^{‡} | |
| e | | ✓ | | ✓ (human cells | ✓ | Electroporate | 0.9%^{§} |
| | | | | electroporated | | from | |
| | | | | separately) | | oocytes^{‡} | |
| f | | | ✓ | ✓ | | iMEF cells; | Negative |
| | | | | | | complete ES | |
| | | | | | | growth | |
| | | | | | | media | |

*Approximately 10⁵ of the following: bone marrow stromal cells, BJ cells, human pre-adipocytes, CD4TLs, human buccal mucosa cells, HeLa cells, MCF-7 cells (all control experiments were conducted separately with each human cell type)
^{‡}Oocytes removed from the electroporate prior to incubation
^{§}Calculated using CD4+ T lymphocytes CD4TLs

Results:

Controls:

Nanog was not detected in human cells from controls “a”, “b”, “c’, and “f”. A small number of human cells from control “d”, in which non-electroporated human cells were exposed for 3 hours to electroporate, expressed the Nanog gene (reprogramming efficiency of about 0.4%; calculated only for CD4TLs). A similarly low number of human cells from control “e” expressed the Nanog gene (0.9% efficiency, calculated only for CD4TLs); in this control, human cells were electroporated in the absence of oocytes and then were exposed to electroporate for 3 hours.

BMSC and BMSC_{GFP}:

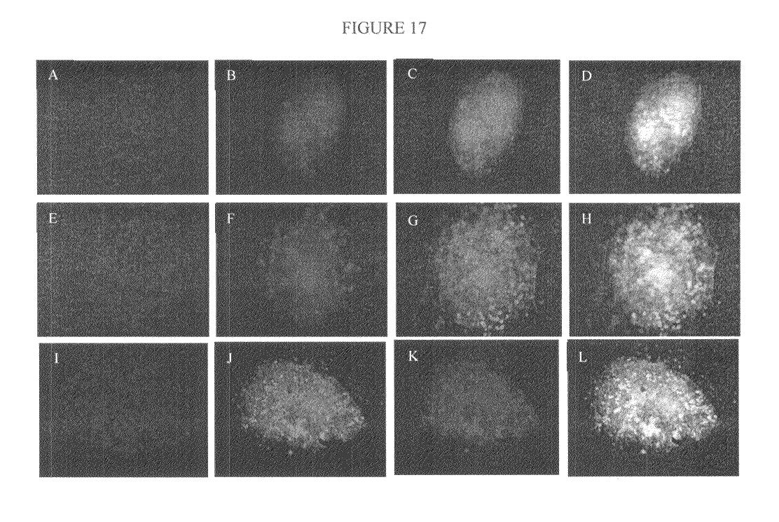

Within one week of co-electroporation with Xenopus laevis oocytes, cells derived from human BMCs co-cultured with iMEF cells expressed the pluripotency-associated transcription factors Oct3/4, SOX-2, Nanog, Rex-1, and SSEA-1 and formed colonies resembling those known to form by iPSC in culture (FIG. 17). In separate studies, BMSC_{GFP} were co-electroporated with Xenopus oocytes and grown on iMEF cells. The resultant cell colonies resembled those of iPSCs and contained cells emitting green fluorescence (data not shown).

BJ Cells:

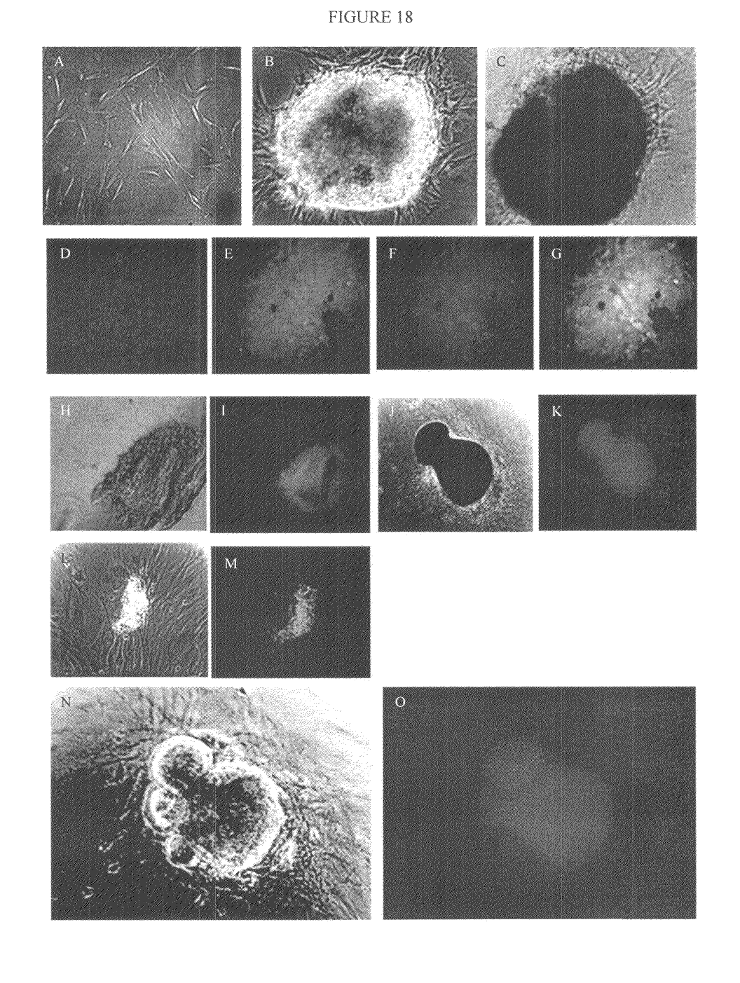

Co-electroporation in the presence of Xenopus oocytes, followed by co-culture on iMEF feeder cells, resulted in reprogramming of BJ cells, evidenced by a high level of alkaline phosphatase activity and resemblance to iPSC in colony morphology and the expression of Oct3/4, Nanog, SOX-2, TRA-1-60, Rex-1, and SSEA-1 (FIG. 18).

HPA Cells-Reprogramming, Cryopreservation, and Trans-Defferentiation:

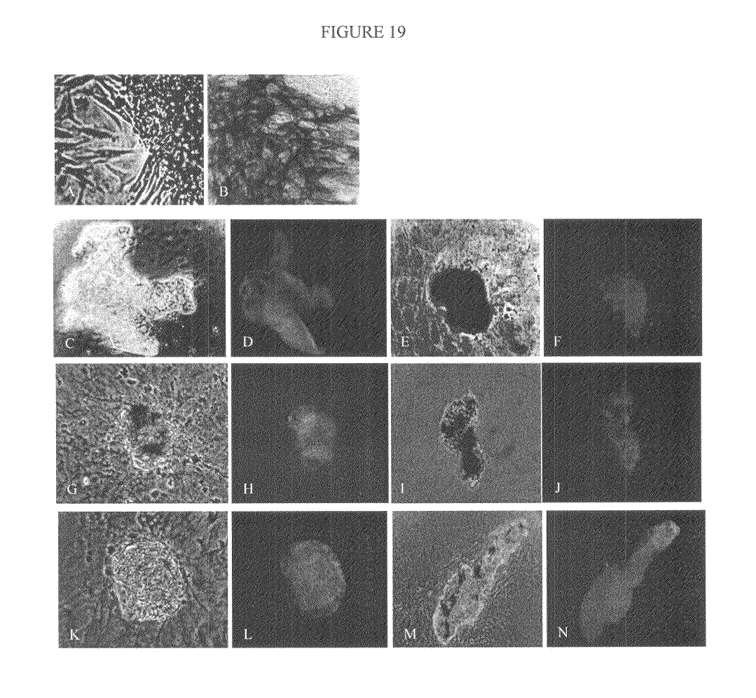

After co-electroporation of HPA and co-culture on feeder cells, the human cells formed colonies morphologically similar to those of iPSC (FIG. 19). The reprogrammed HPA-derived cells displayed strong alkaline phosphatase activity (FIG. 19). The cells in these colonies strongly expressed Oct3/4, Nanog, SOX-2, TRA-1-60, Rex-1, and SSEA-1 (FIG. 19).

One month after cryopreservation of the reprogrammed HPA-derived cells, the reprogrammed cells were thawed, resulting in 78% viability. By day 4 after subculturing on fresh feeder cells the reprogrammed HPA-derived cells formed secondary clusters resembling those formed by iPSC (data not shown).

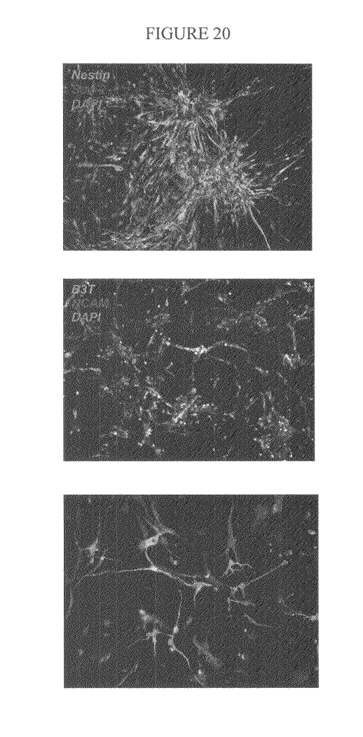

Subculturing cells derived from HPA following co-electroporation in conditions that promote the neural differentiation of embryonic stem cells resulted in formation of cells expressing various immature and mature neural markers including nestin, NCAM, B3T, and GFAP (FIG. 20).

CD4TLs-Reprogramming and Efficiency:

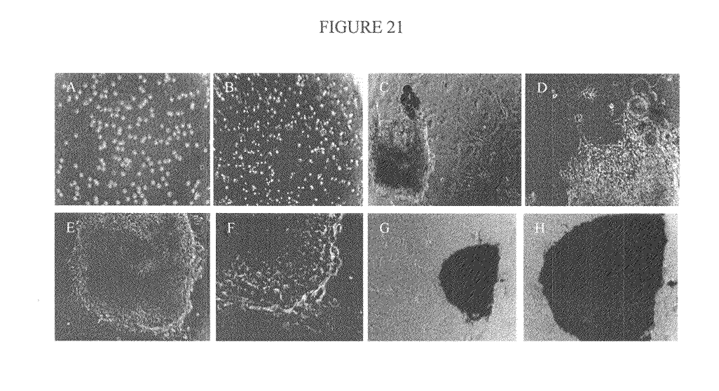

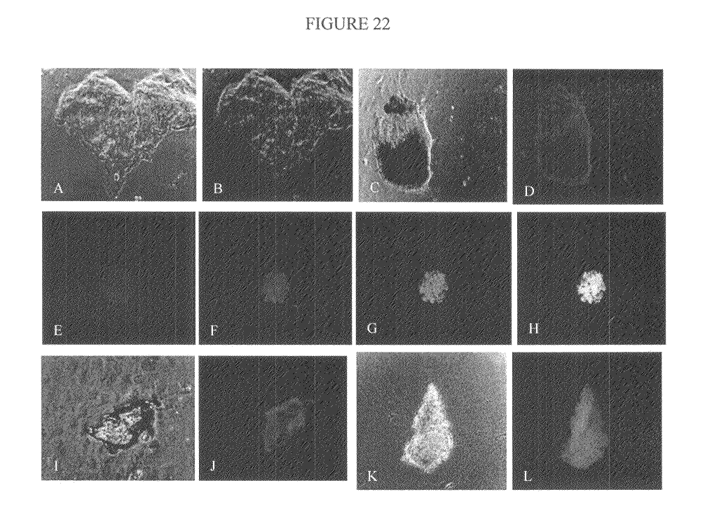

Within 3 to 5 days after transfer to feeder cell layers following co-electroporation with Xenopus laevis oocytes, the human CD4TLs formed colonies similar to those formed by iPSC. Cells in these colonies had high levels of alkaline phosphatase activity (FIG. 21) and strongly expressed Oct3/4, Nanog, SOX-2, TRA-1-60, Rex-1, and SSEA-1 (FIG. 22).

Within 12 to 24 hours after co-electroporation with Xenopus laevis oocytes, the cells derived from human CD4TLs co-cultured with iMEF started to express the Nanog gene. During this time period, single cells and small iPSC-like clusters in which individual cells could be counted were present (data not shown). The proportion of cells expressing Nanog and the total number of cells were counted for calculation of reprogramming efficacy, which was 23.4±3.5%.

Human Buccal Mucosa Cells:

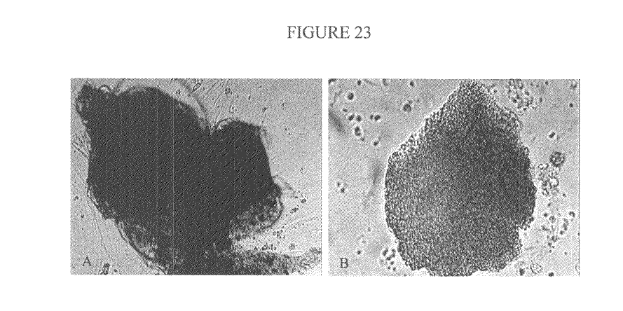

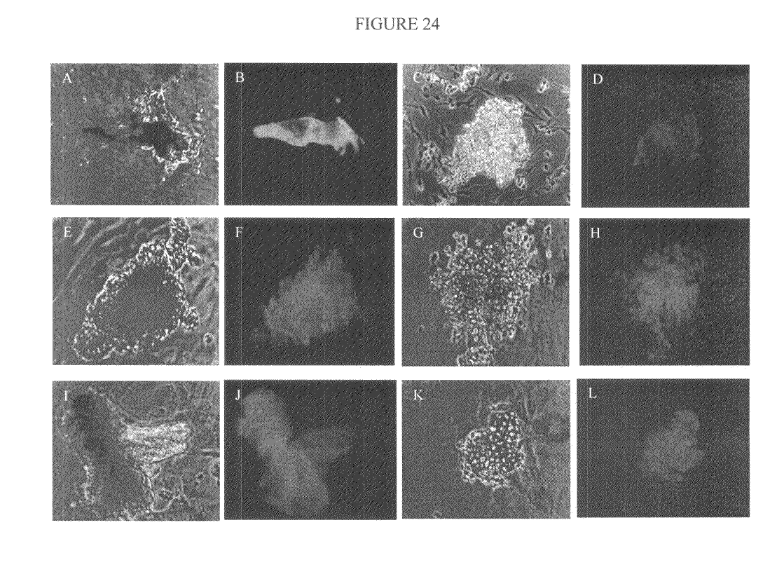

Freshly obtained human buccal mucosa cells, co-electroporated in the presence of Xenopus oocytes and cultured on iMEF and on feeder cell-free StemAdhere™ substrate, gave rise to cells that formed colonies similar to those of iPSC (FIG. 23). Cells in these colonies expressed Oct3/4, Nanog, SOX-2, TRA-1-60, Rex-1, and SSEA-1 (FIG. 24).

HeLa and MCF-7 Cells:

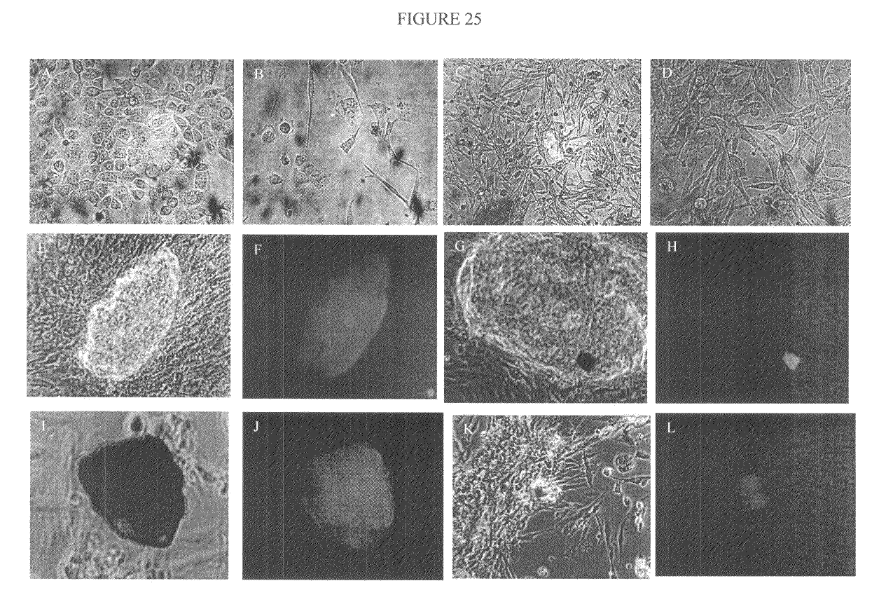

Two human cancer cell lines, HeLa and MCF-7, were subjected to co-electroporation with Xenopus laevis oocytes followed by co-culture on iMEF. The cells derived from co-electroporation of these tumor cells showed partial de-differentiation, with formation of clusters and expression of Oct 3/4 (Hela-derived cells and MCF-7-derived cells) and Nanog (MCF-7-derived cells) (FIG. 25). The cell clusters tended to be smaller than those derived from co-electroporation of non-tumor cells (data not shown).

### Example 12

### Identification of Proteins Involved in Reprogramming

In this study, protein expression from activated Xenopus laevis oocytes was analyzed and compared to protein expression from non-activated Xenopus laevis oocytes in order to identify proteins involved in reprogramming of cells to iPSC-like cells.

Protocol:

Ninety-three proteins were investigated using standard mass spectrometry (MS) analysis. Peptide mixes obtained from in-gel trypsin digest of total protein pools from both activated and non-activated Xenopus laevis oocytes were analyzed using a nanoAcquity UPLC system coupled to a Synapt G2 HDMS mass spectrometer (Waters Corp., Milford, Mass.). Peptides were separated on a 75 μm×100 mm column with 1.7 um C18 BEH particles (Waters) using a 30 min. gradient of 5-35% acetonitrile with 0.1% formic acid at a flow rate of 0.3 μl/min and 35° C. column temperature. For each sample, a data-dependent analysis (DDA) was conducted using a 0.7 sec MS scan followed by MS/MS acquisition on the top three ions with charge greater than one. MS/MS scans for each ion used an isolation window of about 3 Da, a maximum of 2 sec per precursor, and dynamic exclusion for 120 sec within 1.2 Da. DDA data were converted to searchable files using ProteinLynx Global Server 2.4 (Waters Corp.) and searched against the human IPI database v.3.79 (January 2011) using Mascot server 2.2 with the following parameters: maximum one missed cleavage site, carbamidomethylation at Cys residues as fixed modification and Met oxidation, N-terminal acetylation, Asn, Gln deamidation as variable modifications. Precursor ion mass tolerance was set to 20 ppm, while fragment mass tolerance was set to 0.2 Da. Acceptance criteria for protein identification required identification of at least two peptides for each protein with a confidence interval percentage (C1%) over 99.9%, corresponding to a false discovery rate of 0.1%.

Results:

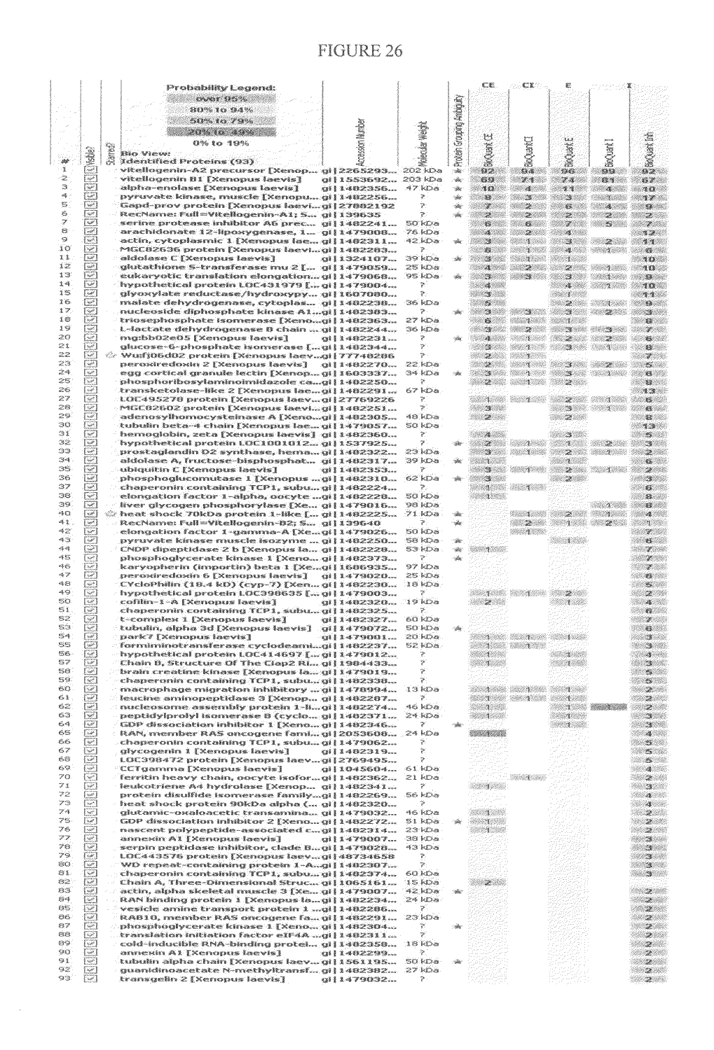

The results of this experiment are presented in FIG. 26. Of the 93 proteins investigated, Gapd-prov protein, prostaglandin D2 synthase, hematopoietic b, phosphoglucomutase 1, hypothetical protein LOC100101274, hypothetical protein LOC398635, vitellogenin-A1, short-VTG-A1, nucleoside diphosphate kinase A1, mg:bb02e05 protein and adenosylhomocysteinase A were identified as proteins present that may be involved in reprogramming of cells to iPSCs.

### Example 13

### Identification of MicroRNAs (miRNAs) Involved in Reprogramming

In this study, the distribution of miRNAs inside and outside activated and non-activated Xenopus laevis oocytes was analyzed in order to identify miRNAs present that may be involved in reprogramming of cells to iPSCs.

Total RNA Isolation:

Total RNA was isolated from activated and non-activated Xenopus laevis oocytes using Trizol® LS reagent (LT cat#10296010) as per manufacturer's protocol.

Endogenous 18s rRNA Gene Expression Assay:

Single-stranded cDNA for 18s rRNA analysis was synthesized using TagMan® reverse transcription reagent and random hexamers as described in the high capacity RNA to cDNA kit protocol (Applied Biosystems cat#4366593). TagMan® qPCR analysis for 18s rRNA was performed using eukaryotic 18s rRNA Assay as described in the Applied Biosystems protocol for pre-developed TagMan® assay reagents (P/N 4323193 REV B). Two sets of qPCR reactions were performed per sample using either 5 μl or 15 μl of RT product.

TagMan® MicroRNA (miRNA) qPCR Analysis:

Single-stranded cDNA for micro RNA profiling was synthesized form samples using the TagMan® MicroRNA Reverse Transcription Kit (P/N 4366593) as described in the Applied Biosystems protocol “TagMan®Small RNA Assays”. Resulting reverse transcription product was used to perform real-time PCR reactions using TagMan® Universal PCR Master Mix, No AmpErase® UNG (P/N 4324018) and microRNA assays. MicroRNA assays were performed to detect 15 miRNAs believed to be involved in animal and human somatic cell reprogramming (See, Anokye-Danso F, Trivedi C M, Juhr D, Gupta M, Cui Z, Tian Y, Zhang Y, Yang W, Gruber P J, Epstein J A, Morrisey E E. Cell Stem Cell, 2011; 8:376-388 and Wilson K D, Venkatasubrahmanyam S, Jia F, Sun N, Butte A J, Wu J C. MicroRNA profiling of human-induced pluripotent stem cells. Stem Cells and Development. 2009; 18(5):749-58). The 15 miRNAs were hsa-miR-17-5p, hsa-nu/r-18a, hsa-miR-92a, hsa-miR-19b-1, hsa-miR-20a, mmu-miR-92a, mmu-miR-93, hsa-miR-367, hsa-miR-372, hsa-miR-373, hsa-miR-106b, hsa-miR-302a, hsa-miR-302b, hsa-miR-302c and hsa-miR-302d. Real-time PCR reactions were performed on a 7900HT system (Applied Biosystems).

Results:

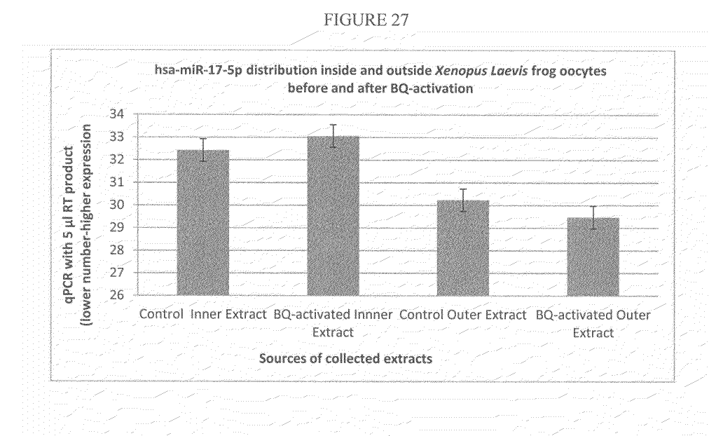

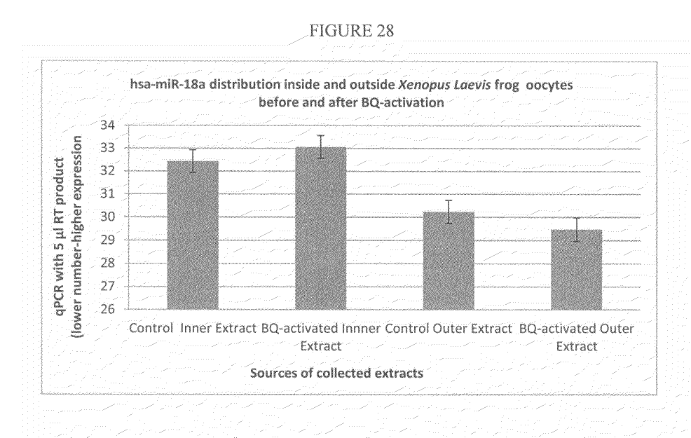

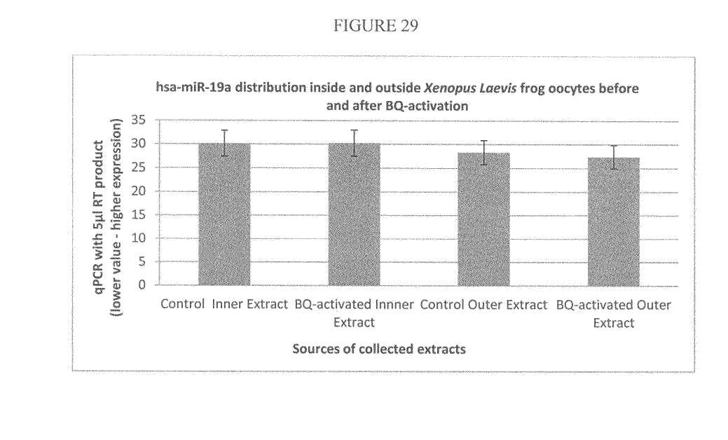

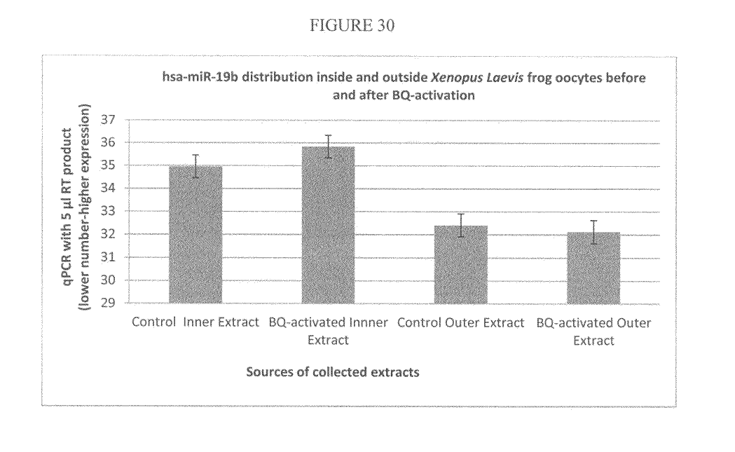

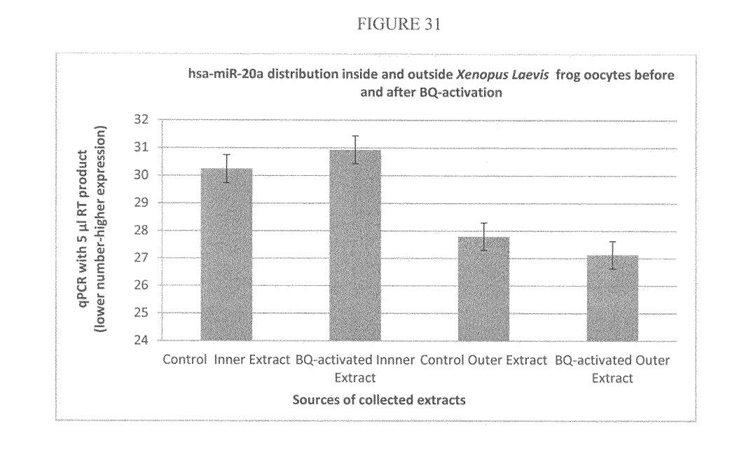

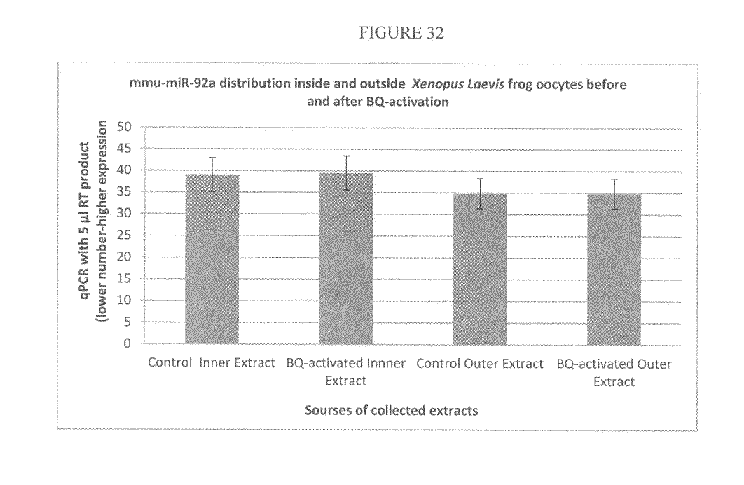

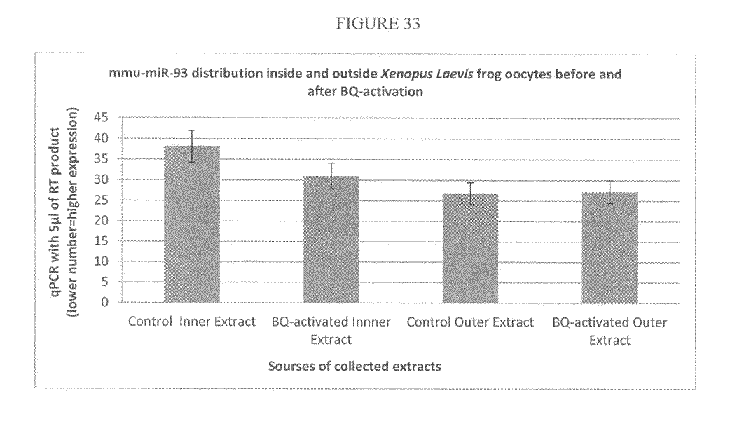

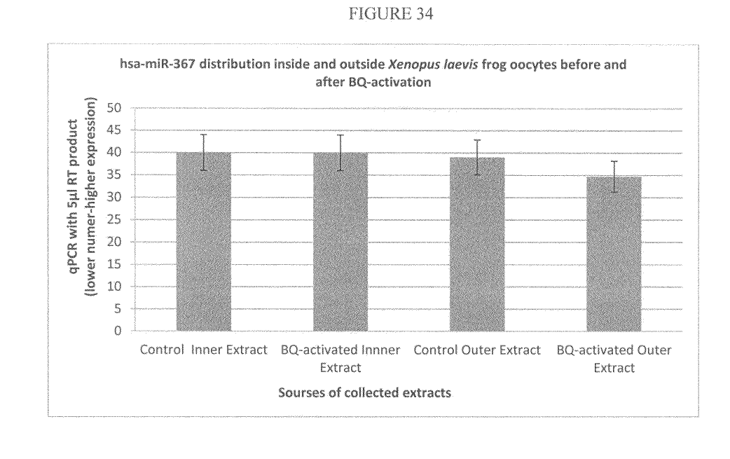

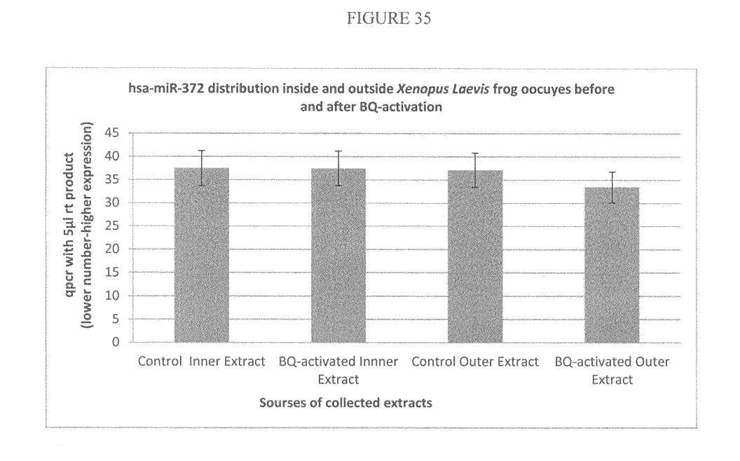

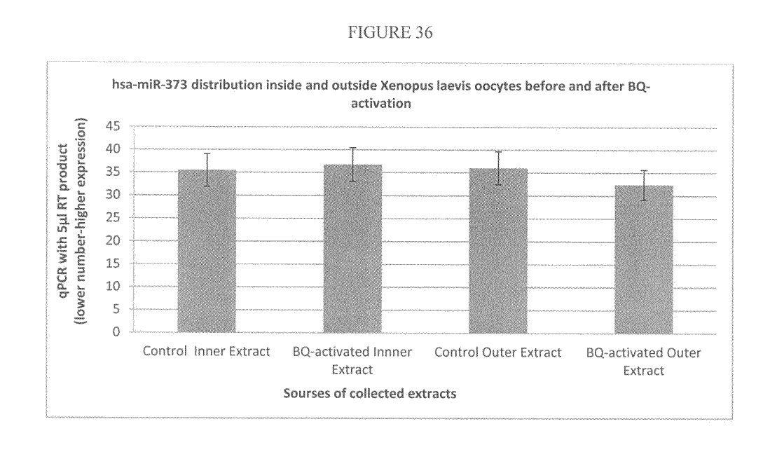

The results of this experiment are presented in FIGS. 27-36. MicroRNAs hsa-miR-1′7-5p, hsa-nu/r-18a, hsa-miR-92a, hsa-miR-19b-1, hsa-miR-20a, mmu-miR-92a, mmu-miR-93, hsa-miR-367, hsa-miR-372 and hsa-miR-373 were positively identified. MicroRNAs hsa-miR-106b, hsa-miR-302a, hsa-miR-302b, hsa-miR-302c and hsa-miR-302d were not detected (data not shown). 18s rRNA was detected in all samples tested (data not shown).

While the present invention has been described with reference to the specific embodiments thereof, it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for preparing a composition comprising extracts of activated amphibian oocytes comprising:
(a) providing a suspension of oocytes harvested from an amphibian, in a buffered oocyte washing solution in an oocyte activation vessel;
(b) applying an electroporation stimulus to the suspended oocytes of (a) in the oocyte activation vessel to produce a suspension of activated oocytes;
(c) combining an aqueous energy solution with the suspension of activated oocytes to form an aqueous suspension;
(d) incubating the aqueous suspension of (c) at an incubation temperature of 16° C. to 20° C., for an incubation time of about 2 to about 4 hours;
(e) partitioning the incubated combination of (d) to obtain a portion external to the incubated activated oocytes (extra-oocyte portion), and an activated oocyte portion that includes the incubated activated oocytes of (d);
(f) separating the extra-oocyte portion and the activated oocyte portion from each other;
(g) filtering the extra-oocyte portion to produce an extra-oocyte composition;
(h) rupturing the activated oocyte portion of (f) comprising a light fraction, a heavy fraction and a cytoplasmic fraction;
(i) separating the cytoplasmic fraction from the light fraction and the heavy fraction to produce a combination of the light fraction and the heavy fraction; and
(j) filtering the combination of (i) to obtain an intra-oocyte composition.

2. The method according to claim 1, wherein the amphibian oocytes are Xenopus laevis oocytes.

3. The method according to claim 1, wherein the activation vessel is selected from the group consisting of a cell culture flask and an electroporation cuvette.

4. The method according to claim 1, wherein the electroporation stimulus is about 100 v/cm to about 200 v/cm at about 25 μF to about 75 μF for about 0.3 msec to about 1.5 msec pulses for about 5 to 10 pulses.

5. The method according to claim 4, wherein the electroporation stimulus is about 125 v/cm at about 50 μF for about 1 msec pulses at about 7 pulses.

6. The method according to claim 1, wherein the incubation temperature is 17° C.

7. The method according to claim 1, wherein the incubation time is 3 hours.

8. The method according to claim 1, wherein the light fraction comprises lipids.

9. The method according to claim 1, wherein the heavy fraction comprises yolk particles.

10. The method according to claim 1, wherein the buffered oocyte washing solution comprises NaCl, HEPES, KCl, MgCl₂, NaHPO₄ and penicillin/streptomycin.

11. The method according to claim 10, wherein the buffered oocyte washing solution is about pH 7.4.

12. The method according the claim 11, wherein the buffered oocyte washing solution comprises about 82.5 mM NaCl, about 5 mM HEPES, about 2.5 mM KCl, about 1 mM MgCl₂, about 1 mM NaHPO₄ and about 0.5% penicillin/streptomycin.

13. The method according to claim 1, wherein the aqueous energy solution comprises creatine phosphate, adenosine-5′-triphosphate (ATP), and MgCl₂.

14. The method according to claim 13, wherein the aqueous energy solution comprises about 7.5 mM creatine phosphate, about 1 mM adenosine-5′-triphosphate (ATP) at pH 7.7, and about 1 mM MgCl₂.

15. The method according to claim 14, wherein the aqueous energy solution is a 1:100 aqueous dilution.

16. The method according to claim 1, wherein the partitioning step is performed by centrifugation.

17. The method according to claim 1, wherein the separating step is performed by a syringe.

18. The method according to claim 1, wherein the filtering step is performed by a filter.

19. The method according to claim 18, wherein the filter has a pore size of about 0.01μ to 1μ.

20. The method according to claim 19, wherein the filter has a pore size of about 0.2μ.

21. The method according to claim 1, wherein the rupturing step is performed by centrifugation.

22. The method according to claim 1, wherein the method further comprises combining the extra-oocyte portion with a mixture comprising a protease inhibitor and a RNase inhibitor.

23. The method according to claim 1, wherein the method further comprises the step of combining the light fraction and the heavy fraction combination with a protease inhibitor and a RNase inhibitor.

24. The method according to claim 1, wherein the composition is a pharmaceutical composition comprising an equal volume of the extra-oocyte composition and the intra-oocyte composition.

25. The method according to claim 24, wherein the pharmaceutical composition further comprises a pharmaceutically acceptable carrier.

26. A pharmaceutical composition prepared by the process of claim 1 comprising:
(a) a protein selected from the group consisting of Gapd-prov, prostaglandin D2 synthetase, hematopoietic b, phosphoglucomutase 1, hypothetical protein LOC100101274, hypothetical protein LOC398635, vitellogenin (VTG)-A1, short-VTG-A1, nucleoside diphosphate kinase A1, mg:bb02e05, adenosylhomocysteinase A, and a combination thereof; and (b) an miRNA selected from the group consisting of hsa-miR-17-5p, hsa-miR-18a, hsa-miR-92a, hsa-miR-19b-1, hsa-miR-20a, mmu-miR-92a, mmu-miR-93, hsa-miR-367, hsa-miR-372, hsa-miR-373, and a combination thereof.

27. A method for treating a disease, disorder, condition or injury characterized by a damaged or cancerous differentiated cell comprising:
(a) preparing a composition by:
(1) providing a suspension of oocytes harvested from an amphibian, in a buffered oocyte washing solution in an oocyte activation vessel;
(2) applying an electroporation stimulus to the suspended oocytes of (1) in the oocyte activation vessel to produce a suspension of activated oocytes;
(3) combining an aqueous energy solution with the suspension of activated oocytes to form an aqueous suspension;
(4) incubating the aqueous suspension of (3) at an incubation temperature of 16° C. to 20° C., for an incubation time of about 2 to about 4 hours;
(5) partitioning the incubated combination of (4) to obtain a portion external to the incubated activated oocytes (extra-oocyte portion), and an activated oocyte portion that includes the incubated activated oocytes of (4);
(6) separating the extra-oocyte portion and the activated oocyte portion from each other;
(7) filtering the extra-oocyte portion to produce an extra-oocyte composition;
(8) rupturing the activated oocyte portion of (6) to produce a light fraction, a heavy fraction and a cytoplasmic fraction;
(9) separating the cytoplasmic fraction from the light fraction and the heavy fraction to produce a combination of the light fraction and the heavy fraction; and
(10) filtering the combination of (9) to obtain an intra-oocyte composition;
(b) formulating a pharmaceutical composition comprising an equal volume of the extra-oocyte composition and the intra-oocyte composition, and optionally a carrier; and
(c) administering a therapeutic amount of the pharmaceutical composition of (b) to a subject in need thereof,
wherein the therapeutic amount is effective to reprogram the damaged or cancerous cells into iPSC-like cells capable of differentiating into cells capable of repairing the damaged or cancerous cells, thereby treating the disease, disorder, injury or condition.

28. The method according to claim 27, wherein the amphibian oocytes are Xenopus laevis oocytes.

29. The method according to claim 27, wherein the activation vessel is selected from the group consisting of a cell culture flask and an electroporation cuvette.

30. The method according to claim 27, wherein the electroporation stimulus is about 100 v/cm to about 200 v/cm at about 27 μF to about 75 μF for about 0.3 msec to about 1.5 msec pulses for about 5 to 10 pulses.

31. The method according to claim 30, wherein the electroporation stimulus is about 125 v/cm at about 50 μF for about 1 msec pulses at about 7 pulses.

32. The method according to claim 27, wherein the incubation temperature is 17° C.

33. The method according to claim 27, wherein the incubation time is 3 hours.

34. The method according to claim 27, wherein the light fraction is comprised of lipids.

35. The method according to claim 27, wherein the heavy fraction is comprised of yolk particles.

36. The method according to claim 27, wherein the buffered oocyte washing solution comprises NaCl, HEPES, KCl, MgCl₂, NaHPO₄ and penicillin/streptomycin.

37. The method according to claim 36, wherein the buffered oocyte washing solution is about pH 7.4.

38. The method according the claim 37, wherein the buffered oocyte washing solution comprises about 82.5 mM NaCl, about 5 mM HEPES, about 2.5 mM KCl, about 1 mM MgCl₂, about 1 mM NaHPO₄ and about 0.5% penicillin/streptomycin.

39. The method according to claim 27, wherein the aqueous energy solution comprises creatine phosphate, adenosine-5′-triphosphate (ATP), and MgCl₂.

40. The method according to claim 39, wherein the aqueous energy solution comprises about 7.5 mM creatine phosphate, about 1 mM adenosine-5′-triphosphate (ATP) at pH 7.7, and about 1 mM MgCl₂.

41. The method according to claim 40, wherein the aqueous energy solution is a 1:100 aqueous dilution.

42. The method according to claim 27, wherein the partitioning step is performed by centrifugation.

43. The method according to claim 27, wherein the separating step is performed by a syringe.

44. The method according to claim 27, wherein the filtering step is performed by a filter.

45. The method according to claim 44, wherein the filter has a pore size of about 0.01μ to 1μ.

46. The method according to claim 45, wherein the filter has a pore size of about 0.2μ.

47. The method according to claim 27, wherein the rupturing step is performed by centrifugation.

48. The method according to claim 27, wherein the administering is parenterally.

49. The method according to claim 48, wherein the administering is selected from the group consisting of an intraperitoneal injection, a subcutaneous injection, or an intramuscular injection.

50. The method according to claim 49, wherein the injection is an intraperitoneal injection.

51. The method according to claim 27, wherein the differentiated cell is selected from the group consisting of a bone marrow cell, a fibroblast cell, an adipocyte, a peripheral blood CD4+ T-lymphocyte, a buccal cell, a cancer cell, and a senescent cell.

52. The method according to claim 51, wherein the cancer cell is selected from the group consisting of a cervical carcinoma cell, a breast adenocarcinoma cell and a melanoma cell.

53. The method according to claim 25, wherein the disease, disorder, condition or injury is selected from the group consisting of cancer, traumatic brain injury, traumatic alopecia, skin wrinkling and aging.

54. The method according to claim 51, wherein the cancer is selected from the group consisting of melanoma, cervical carcinoma and breast adenocarcinoma.

55. The method according to claim 52, wherein the cancer is melanoma.

56. The method according to claim 27, wherein the method further comprises combining the extra-oocyte portion with a protease inhibitor and a RNase inhibitor.

57. The method according to claim 27, wherein the method further comprises the step of combining the light fraction and the heavy fraction combination with a protease inhibitor and a RNase inhibitor.
